(19) United States
(12) Patent Application Publication
(10) Pub. No.: US 2023/0274827 A1
(43) Pub. Date: Aug. 31, 2023

(54) PHARMACY ROBOT DEVICE, METHOD AND SYSTEM THEREOF

(71) Applicant: Xiamen Brana Design Co., Ltd, Xiamen, Fujian Province (CN)

(72) Inventor: Zhongtang WANG, Xiamen (CN)

(21) Appl. No.: 18/015,052

(22) PCT Filed: Feb. 26, 2021
(86) PCT No.: PCT/CN2021/078173

§ 371 Date: Jan. 6, 2023

(30) Foreign Application Priority Data
Jul. 6, 2020 (CN) 202010679136.5
Jan. 4, 2021 (CN) 202110073502.7

(51) Int. Cl. G16H 40/63; G16H 40/20; G16H 20/13; A61J 7/00

(52) U.S. Cl. CPC G16H 40/63; G16H 40/20; G16H 20/13; A61J 7/0076

(57) ABSTRACT

A pharmacy robot device includes a single pharmacy robot and a pharmacy robot complex formed by connecting a plurality of single pharmacy robots, which can perform a function of drug replenishment, delivery, and inventory, combine with an artificial intelligent verification system and intelligent diagnosis system to realize pharmacy as physician services, pharmacy as pharmacist services and pharmacy as pharmacy management services. Moreover, a plurality of single pharmacy robots can be connected in series, in parallel, or in series and parallel so as to increase a category and quantity of stored drugs, and serve a plurality of clients in parallel.

### BACKGROUND OF THE PRESENT INVENTION

### Field of Invention

This invention relates to a field of medical robots, and more particularly relates to a pharmacy robot device, service method and system for the pharmacy robot.

### Description of Related Arts

Due to some common diseases, frequently-occurring diseases, and chronic diseases, patients often need to go to a hospital to see a doctor or go to a pharmacy to buy medicines.

In some cases, such as remote smaller towns and villages, where the hospital is far away and the pharmacy is relatively scarce, it may be inconvenient for a patient to obtain medical care and medical services. On the one hand, operating cost of the hospital is very expensive, income brought by the patients with common diseases, frequently-occurring diseases and chronic diseases is not much, so that it is difficult to maintain the operation of the hospital for a long time. On the other hand, a higher rent and labor cost of the pharmacy has pushed up a price of medical services and increased a financial burden of the patients.

Compared with intractable diseases and diseases requiring surgical treatment, a diagnosis and treatment of those common diseases, frequently-occurring diseases and chronic diseases can be highly standardized, and a drug for symptomatic treatment is relatively constant. It is completely possible to deploy simple pharmacy robots with functions of doctor's consultation, pharmacist review, drug distribution, and pharmacy service to remote smaller towns and villages, so as to efficiently serve the patients and reduce the operating cost of the pharmacy.

### SUMMARY OF THE PRESENT INVENTION

### Technical Problem to be Solved

An objective of the present invention is to provide a pharmacy robot device, which can perform a basic function of pharmacy, combine artificial intelligent (AI) verification system and intelligent diagnosis system, and realize pharmacy as physician service, pharmacy as pharmacist service and pharmacy as pharmacy management service, so as to serve the patients efficiently and reduce the operating cost of the pharmacy.

### Technical Solutions

A pharmacy robot device according to one embodiment of the present invention comprises a single pharmacy robot, and a pharmacy complex including a plurality of the single pharmacy robots.

A single pharmacy robot according to one embodiment of the present invention comprises a chassis and a robot body, the chassis can be configured to support the robot body, the robot body comprises a shell, a drug store, a push rod, a conveyor belt, an induction door, a display, a smart speaker, a visual sensor, a central data storage processor, and a wireless communication equipment.

A pharmacy complex according to one embodiment of the present invention comprises a multi-unit pharmacy robot and a medicine delivery device, the medicine delivery device can join the multi-unit pharmacy robot into a unitary structure.

The multi-unit pharmacy robot according to one embodiment of the present invention comprises a series single pharmacy robot, a parallel single pharmacy robot, and a series-parallel single pharmacy robot. The series single pharmacy robot includes a plurality of single pharmacy robots arranged in front and back, the parallel single pharmacy robot includes a plurality of single pharmacy robots arranged and combined in left and right, and the series and parallel single pharmacy robot includes a plurality of single pharmacy robots arranged in a front-rear series and a left-right parallel, and a drug delivery device can connect the single pharmacy robots in series with a plurality of conveyor belts.

Preferably, the shell comprises a central shell and two edge shells, the central shell includes a drug store, the edge shells are arranged on both sides of the central shell, and the edge shells includes a push rod, and a rear side of the edge shell and a rear side of the central shell can be connected through a rotating shaft, and a lock can be provided between a front side of the edge shell and a front side of the central shell.

Preferably, the drug store comprises a plurality of medicine cabinets, the medicine cabinets can be fixed in the central shell, the conveyor belt can be arranged at a bottom of a gap between the medicine cabinets. The medicine cabinet can be further divided into a plurality of medicine boxes arranged in a vertical and/or horizontal array.

Preferably, the push rod includes a plurality of Cartesian coordinate robots which can be fixed to the edge shells on both sides through a frame.

Preferably, the conveyor belt can also be connected with an intelligent controlled elevator, so that when the Cartesian robot end effector pushes out a medicine, the intelligent controlled elevator moves synchronously, and the medicine can leave the medicine box and fall on the conveyor belt.

According to one embodiment of the present invention, a medicine delivery device comprises a conveying equipment, a collection bin, and a lifting equipment. The conveying equipment can be connected to the conveyor belt of the single pharmacy robot, the collecting bin can be connected to the conveying equipment, and the lifting equipment can be connected to the conveying equipment or the collection bin.

Preferably, the conveying equipment includes a crawler bearing equipment, a slide way equipment, and/or an unmanned vehicle equipment configured to connect across a plurality of the single pharmacy robots in parallel or in series and parallel.

A method for running a pharmacy robot according to one embodiment of the present invention comprises a drug sales method, a supplementary drug method, a drug disk method, a conjoined method, and a grid deployment method.

The drug sales method process 100 according to one embodiment of the present invention comprises a plurality of steps as follows: 110: the pharmacy robot is as a physician service; 120: the pharmacy robot is as a pharmacist service; 130: the pharmacy robot confirms a patient, reminds the patient through a human-computer interaction whether or to buy a medicine, if so, entering a next step; 140: the patient pays a fee; 150: the Cartesian coordinate robot picks up the medicine; 160: the induction door opens, and the patient gets the medicine; 170: the pharmacy robot is as a pharmacy management service.

The supplementary drug method process 200 according to one embodiment of the present invention comprises a plurality of steps as follow: 210: a cloud server statistics data; 220: a supply chain system prepares a drug according to a statistical data; 230: a logistics system transports the drug to the pharmacy robot; 240: the pharmacy robot opens a drug store, medicine cabinet and medicine box; 250: a replenishment personnel fills up all the medicine boxes; 260: the pharmacy robot returns to a normal status.

The conjoined method process according to one embodiment of the present invention includes a series connection, a parallel connection and a series-parallel connection configured to increase the categories and quantities of medicines stored in the drug stores. The series connection can be realized by a front and rear arrangement and combination of a plurality of single pharmacy robots, the parallel connection can be realized by a left and right arrangement and combination of a plurality of single pharmacy robots, the series-parallel connection can be composed of a plurality of the single pharmacy robots arranged in series in front and rear and arranged in parallel on left and right.

The grid deployment method process according to one embodiment of the present invention includes a site selection, a pharmacy kiosk configuration, a selection of a single pharmacy robot or a pharmacy complex, and a drug category selection. A data sharing between outlets in a service area, as well as with other pharmacies, hospital pharmacies, and suppliers, is convenient for clients to obtain drug information and obtain drugs and services through multiple channels.

Preferably, the replenishment personnel of the supplementary drug method process can be implemented by a replenishment robot.

A system according to one embodiment of the present invention comprises a drug screening system, an artificial intelligent verification system, an intelligent diagnosis and treatment system, a drug delivery system, a drug sales system, a drug management system, a third-party support system, a database management system, a data encryption system, a medical information sharing and supervision system, a drug circulation traceability system, an electronic prescription system, a cloud server, a 5G network and a settlement system configured to realize a full-process pharmacy service of a single pharmacy robot or pharmacy robot complex, which including pharmacy as doctor service, pharmacy as pharmacist service and pharmacy as pharmacy management service.

### Beneficial Effects

(1) The storage of medicines is more intensive and space is more fully utilized. For example, a number of medicine boxes in a drug store of a single pharmacy robot device can be up to 900, and each medicine box can hold 5˜10 boxes of medicines, basically realizing the storage of at least 600 medicine categories, at least 3,000 boxes drugs, if connecting multiple single pharmacy robots in series, parallel or series-parallel, the drug store can cover all commonly drugs and medical supplies.

(2) The single pharmacy robot or pharmacy robot complex has an overall complete function, as well as a simple structure, a compact design, and a convenient maintenance. For example, a coordinate robot and an end effector are simple and durable with a high reliability and good flexibility, a chassis with universal wheels is convenient to move, the shell structure can be opened and locked with an intuitive vision.

(3) The cost of pharmacy robot is low because its components are in sufficient supply in market.

(4) A function of pharmacy as doctor service, pharmacy as pharmacist service and pharmacy as pharmacy management service is realized, which makes the pharmacy become more intelligence, saves time and cost for patients, and improves more thoughtful and comprehensive medical treatment, medicine and follow-up services.

(5) Cooperate with a medicine kiosk to set up a smart stethoscope, a smart percussion hammer, an automatic electrocardiograph, and other simple and easy-to-use medical inspection equipment, which is convenient for patients saving a need for a hospital.

(6) Setting up and deploying pharmacy robots and kiosks has low difficulty and low threshold, thereby saving social resources and reducing pharmacy operating costs.

### BRIEF DESCRIPTION OF THE DRAWINGS

In order to illustrate a technical solution of an embodiment of the present invention more clearly, the following brief introduces an accompanying drawing used in a description of the embodiment. Obviously, the drawings in the following description are only some embodiments of the present invention. For those skilled in an art, other drawings can also be obtained from these drawings without any creative effort.

FIG. 1 is a schematic structural diagram of an outline of a single pharmacy robot according to a preferred embodiment of the present invention;

FIG. 2 is a schematic diagram of a layout of a single pharmacy robot drug store according to the embodiment of the present invention;

FIG. 3 is a schematic structural diagram of a single pharmacy robot medicine box according to the embodiment of the present invention;

FIG. 4 is a schematic structural diagram of a single pharmacy robot Cartesian robot equipment according to the embodiment of the present invention;

FIG. 5 is a schematic structural diagram of a single pharmacy robot Cartesian robot end effector according to the embodiment of the present invention;

FIG. 6 is a schematic structural diagram of a single pharmacy robot induction door according to the embodiment of the present invention;

FIG. 7 is a schematic diagram of a layout of a medicine kiosk according to the embodiment of the present invention;

FIG. 8 is a schematic diagram of a series connection of a plurality of single pharmacy robots according to the embodiment of the present invention;

FIG. 9 is a schematic diagram of a series—parallel connection of a plurality of single pharmacy robots according to the embodiment of the present invention;

FIG. 10 is a schematic diagram of a plurality of single pharmacy robots in parallel with a crawler bearing and conveying structure according to the embodiment of the present invention;

FIG. 11 is a schematic diagram of a plurality of single pharmacy robots in series-parallel with a crawler bearing and conveying structure according to the embodiment of the present invention;

FIG. 12 is a schematic diagram of a plurality of single pharmacy robots in parallel with a single-track unmanned vehicle according to the embodiment of the present invention;

FIG. 13 is a schematic diagram of a plurality of single pharmacy robots in parallel with a double-track unmanned vehicle according to the embodiment of the present invention;

FIG. 14 is a schematic diagram of a plurality of single pharmacy robots in parallel with a trackless unmanned vehicle according to the embodiment of the present invention;

FIG. 15 is a schematic diagram of a plurality of single pharmacy robots in parallel with a chute according to the embodiment of the present invention;

FIG. 16A is a schematic diagram of a plurality of single pharmacy robots in parallel with a middle slide of a chute according to the embodiment of the present invention;

FIG. 16B is a schematic diagram of a plurality of single pharmacy robots in parallel with a left slide of a chute according to the embodiment of the present invention;

FIG. 16C is a schematic diagram of a plurality of single pharmacy robots in parallel with a right slide of a chute according to the embodiment of the present invention;

FIG. 17 is a schematic diagram of a plurality of single pharmacy robots in parallel with a pulley rope lifting crawler device according to the embodiment of the present invention;

FIG. 18A is a schematic diagram of a plurality of single pharmacy robots in parallel with a screw shaft axially lifting crawler device according to the embodiment of the present invention;

FIG. 18B is a schematic diagram of a plurality of single pharmacy robots in parallel with a screw nut axially lifting crawler device according to the embodiment of the present invention;

FIG. 19 is a schematic diagram of a plurality of single pharmacy robots in parallel with a hydraulic scissor lifting crawler device according to the embodiment of the present invention;

FIG. 20 is a schematic diagram of a plurality of single pharmacy robots in parallel with a screw axial shaft lifting collecting bin according to the embodiment of the present invention.

### DETAILED DESCRIPTION OF THE PREFERRED EMBODIMENT

In order to make an objective, technical solution and advantage of the present invention clearer, the present invention will be further described in detail below with reference to the accompanying drawings and embodiments. It should be understood that a specific embodiment described herein is only used to explain the present invention, not to limit the present invention.

It should be noted that an orientation term such as up, down, left, and right in the embodiment is only a relative concept to each other or refer to a normal use state of a product, and should not be regarded as limiting.

A pharmacy robot device according to a preferred embodiment of the present invention includes a single pharmacy robot, and a plurality of single pharmacy robots is connected to form a pharmacy robot complex. The single pharmacy robot includes a chassis and a robot body, the chassis carries the robot body, the robot body can be provided with a shell, a drug store, a push rod device, a conveyor belt, an induction door, a display, a smart speaker, a visual sensor, a central data storage, a processor, and a wireless communication equipment. The pharmacy robot complex includes a multi-unit pharmacy robot and a medicine delivery device which joins the multi-unit pharmacy robot into an integral structure.

The multi-unit pharmacy robot in the preferred embodiment of the present invention includes a series unit pharmacy robot, a parallel unit pharmacy robot, and a series-parallel unit pharmacy robot. The series unit pharmacy robot comprises a plurality of the single pharmacy robots arranged in in front and rear series, the parallel unit pharmacy robot comprises a plurality of the single pharmacy robots arranged in left and right parallel, and the series-parallel unit pharmacy robot comprises a plurality of the single pharmacy robots arranged in front and rear series and left and right parallel. The medicine delivery device can be connected to a plurality of conveyor belts of the single pharmacy robot in series.

The medicine delivery device according to the preferred embodiment of the present invention includes a conveying device, a collection bin, and a lifting device. The conveying device can be connected to the conveyor belt of the single pharmacy robot, the collecting bin can be connected to the conveying device, and the lifting device can be connected to the conveying device or the collection bin.

A method for running a pharmacy robot according to the embodiment of the present invention comprises a drug sales method, a supplementary drug method, a drug disk method, a conjoined method, and a grid deployment method.

The drug sales method process 100 according to the embodiment of the present invention comprises a plurality of steps as follows: 110: the pharmacy robot is as a physician service; 120: the pharmacy robot is as a pharmacist service; 130: the pharmacy robot confirms a patient, reminds the patient through a human-computer interaction whether or to buy a medicine, if so, entering a Next step; 140: the patient pays a fee; 150: the Cartesian coordinate robot picks up the medicine; 160: the induction door opens, and the patient gets the medicine; 170: the pharmacy robot is as a pharmacy management service.

The supplementary drug method process 200 according to the embodiment of the present invention comprises a plurality of steps as follow: 210: a cloud server statistics data; 220: a supply chain system prepares a drug according to a statistical data; 230: a logistics system transports the drug to the pharmacy robot; 240: the pharmacy robot opens a drug store, medicine cabinet and medicine box; 250: a replenishment personnel fills up all the medicine boxes; 260: the pharmacy robot returns to a normal status.

The conjoined method process according to the embodiment of the present invention includes a series connection, a parallel connection and a series-parallel connection configured to increase the categories and quantities of medicines stored in the drug stores. The series connection can be realized by a front and rear arrangement and combination of a plurality of single pharmacy robots, the parallel connection can be realized by a left and right arrangement and combination of a plurality of single pharmacy robots, the series-parallel connection can be composed of a plurality of the single pharmacy robots arranged in series in front and rear and arranged in parallel on left and right.

The grid deployment method process according to the embodiment of the present invention includes a site selection, a pharmacy kiosk configuration, a selection of a single pharmacy robot or a pharmacy complex, and a drug category selection. A data sharing between outlets in a service area, as well as with other pharmacies, hospital pharmacies, and suppliers, is convenient for clients to obtain drug information and obtain drugs and services through multiple channels.

A system according to the embodiment of the present invention comprises a drug screening system, an artificial intelligent verification system, an intelligent diagnosis and treatment system, a drug delivery system, a drug sales system, a drug management system, a third-party support system, a database management system, a data encryption system, a medical information sharing and supervision system, a drug circulation traceability system, an electronic prescription system, a cloud server, a 5G network and a settlement system configured to realize a full-process pharmacy service of a single pharmacy robot or pharmacy robot complex.

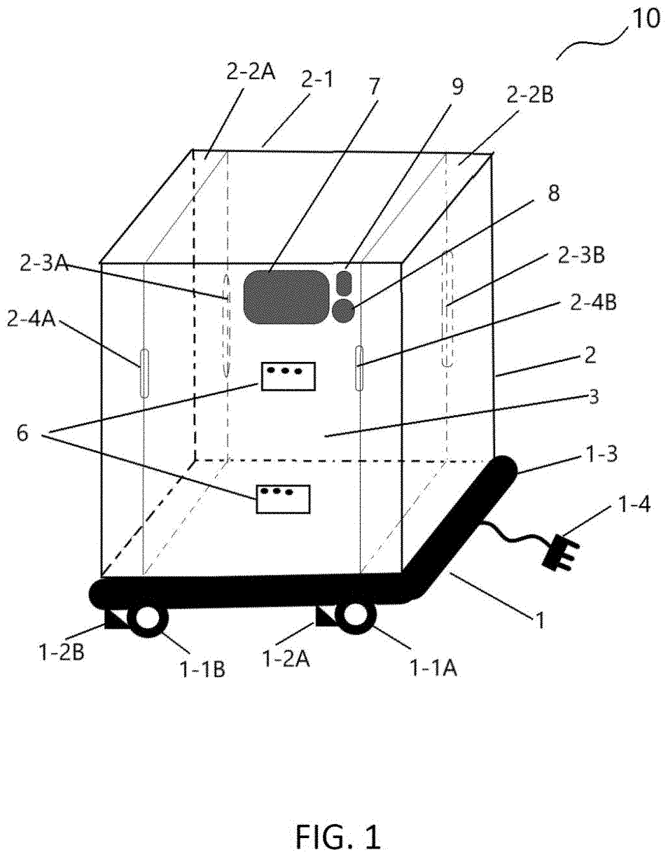

Referring to FIGS. 1 to 20, a pharmacy robot device according to the embodiment of the present invention can comprise a single pharmacy robot 10, and a pharmacy robot complex 20 which including a plurality of the single pharmacy robots.

As shown in FIGS. 1 to 6, a single pharmacy robot 10 according to the embodiment of the present invention can comprise a chassis 1, a shell 2, a drug store 3, a push rod 4, a conveyor belt 5, an induction door 6, a display 7, a smart speaker 8, a visual sensor 9, a central data storage processor and a wireless communication equipment, the central data storage processor can realize a data link and intelligent control of the chassis 1, the shell 2, the drug store 3, the push rod 4, the conveyor belt 5, the induction door 6, the display 7, the smart speaker 8 and the visual sensor 9 through the wireless communication equipment.

Referring to FIG. 1, the chassis 1 can comprise a base 1-3, a four-universal wheel 1-1, a brake 1-2 and a power supply and interface 1-4, the power supply can include an external power supply and a battery to provide power. The shell 2 can comprise a central shell 2-1, a left edge shell 2-2A and a right edge shell 2-2B, the central shell 2-1 includes a drug store 3, the left edge shell 2-2A and right edge shell 2-2B A can include the push rod 4 respectively. The left edge shell 2-2A and the right edge shell 2-2B can be respectively connected with the center shell 2-1 at a rear side through a left shaft 2-3A and a right shaft 2-3B, and the left edge shell 2-2A and the right edge shell 2-2B can be flipped along a left rotation axis 2-3A and a right rotation axis 2-3B respectively. A left lock 2-4A and a right lock 2-4B can be respectively arranged between a side and the front side of the central shell 2-1. The induction door 6, the display 7, the smart speaker 8, and the visual sensor 9 can be arranged on a front side of the central shell 2-1.

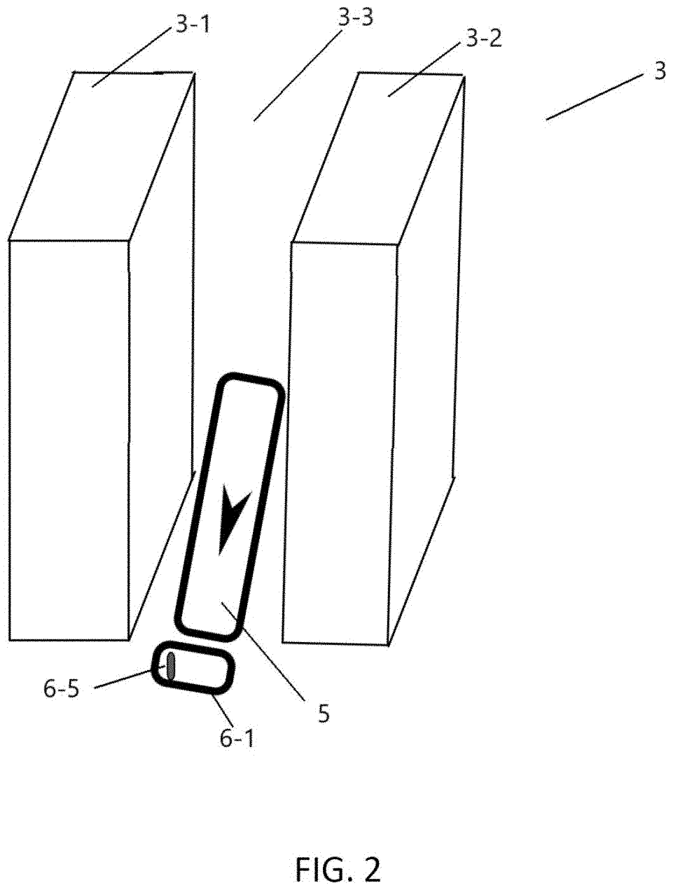

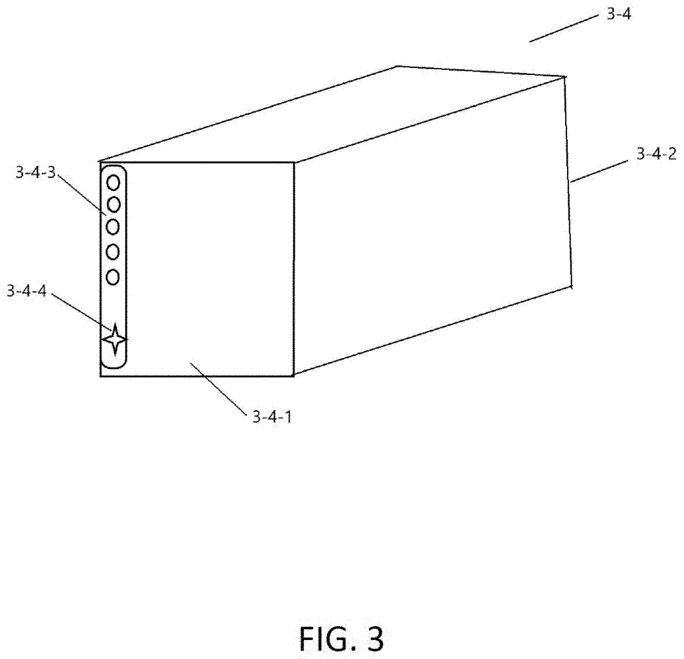

Referring to FIGS. 2 and 3, a drug store 3 according to the embodiment of the present invention can comprise a left medicine cabinet 3-1 and a right medicine cabinet 3-2, which can be fixed in the central shell 2-1 with a gap 3-3 between the left medicine cabinet 3-1 and the right medicine cabinet 3-2 is not less than 15 cm. A conveyor belt 5 can be arranged at a bottom of the gap 3-3, and fixed in the center shell 2-1. A temporary storage concave plate 6-1 for receiving medicines can be arranged at a front end of the conveyor belt 5, which can be fixed in the central shell 2-1. Moreover, the left medicine cabinet 3-1 and the right medicine cabinet 3-2 can be further divided into a plurality of medicine boxes 3-4 in vertically and horizontally arrangement, with an adjustable width and height. The medicine boxes 3-4 can be arranged horizontally, each medicine box 3-4 can store one medicine category, each medicine boxes 3-4 can be positioned by coordinates (X, Y), where X is a number of a horizontal grid, and Y is a number of a vertical grid. Each box of medicines stored in each medicine box 3-4 can also be positioned by coordinates (X, Y, Z), where X is the number of the horizontal grids, Y is the number of the vertical grids, and Z is an ordinal number in the medicine box 3-4. After an ordinal drug in a front row are taken out, a position of the ordinal drugs in a rear row can move forward, and reorder accordingly.

Referring to FIG. 3, the left medicine cabinet 3-1 can be taken as an example to illustrate a structural feature of the medicine box 3-4. In the embodiment of the present invention, the medicine box 3-4 can be open on both sides, a side wall of a first opening 3-4-1 can be provided with a first light strip 3-4-3 with no less than five diode lamps with chip and a first indicator light 3-4-4. The first light strip 3-4-3 can include a red-light bead and a green-light bead. A number of flashing green-light bead is a number of existing drugs, and s number of flashing red-light bead is a number of vacant drug or replenish number of medicine boxes. The first indicator light 3-4-4 can be as an out-of-stock indicator light. A replenishment personnel scan and identify a medicine through the visual sensor 9, the first indicator light 3-4-4 of a corresponding medicine box 3-4 can instruct the replenishment personnel to find the medicine box 3-4 to be replenished in time, and according to the number of red-light beads flashing, know the number of replenishing categories in time. A second opening 3-4-2 close to a gap 3-3 can be a outlet of the medicine box 3-4, wherein a integer of a long diameter cm of the medicine box 3-4 divided by a long diameter cm of a medicine packaging box is the maximum number of medicine boxes that the medicine box 3-4 can accommodate, and a remainder can be used to calculate an extra working distance of the push rod 4.

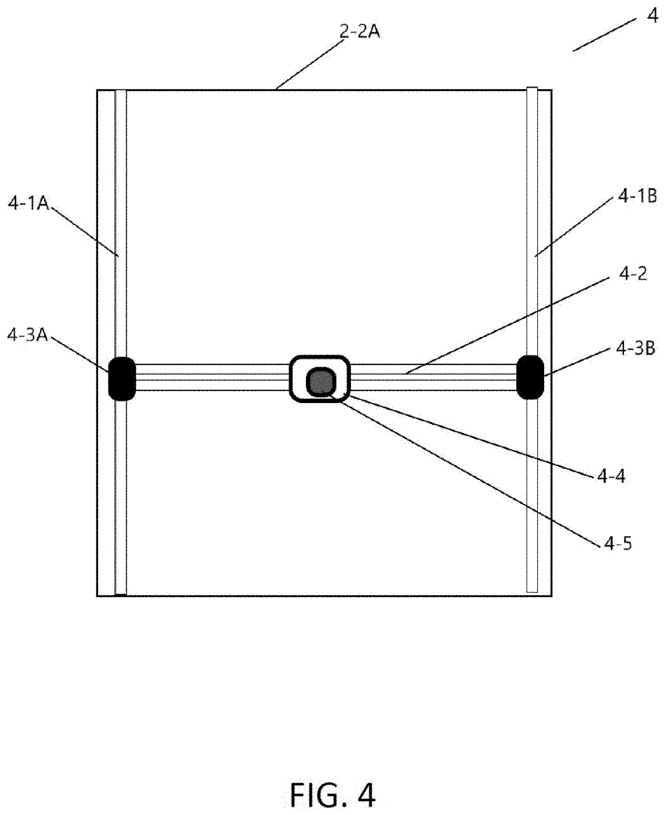

Referring to FIG. 4, the left edge shell 2-2A and the right edge shell 2-2B of an embodiment of the present invention can include a push rod 4 respectively, and the push rod 4 of the left edge shell 2-2A can be taken as an example to explain its structural composition and working principle. The main structure of the push rod 4 includes a Cartesian coordinate robot. The Cartesian coordinate robot can be fixed on the left edge shell 2-2 A through a left vertical frame Y axis 4-1A, a right vertical frame Y axis 4-1B and a horizontal frame X axis 4-2, the left vertical frame Y-axis 4-1A, the right vertical frame Y-axis 4-1B and the horizontal frame X-axis 4-2 include a sliding rail, a left intelligently control drive motor 4-3A and a right-side intelligently control drive motor 4-3B can move in a vertical direction along the left vertical frame Y-axis 4-1A and the right vertical frame Y-axis 4-1B lead screw respectively, a middle intelligently control drive motor 4-4 can move left and right along a lead screw of the horizontal frame X axis 4-2, there is an end effector 4-5 at the Z axis direction.

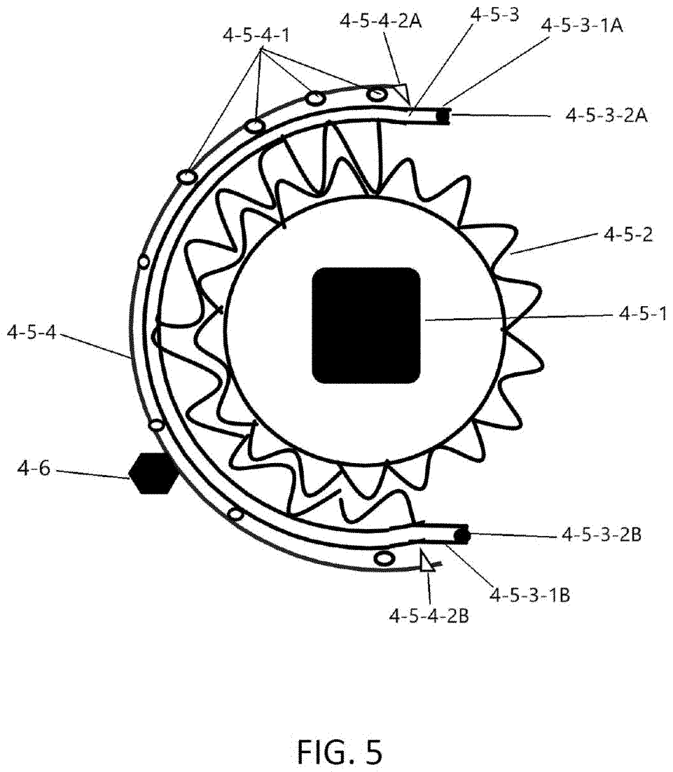

As shown in FIG. 5, the end effector 4-5 of an embodiment of the present invention can be connected with the intermediate intelligent control drive motor 4-4 through a fixing member 4-6. The end effector 4-5 comprises a servo motor 4-5-1, a reducer, a controller, a gear 4-5-2, a rack 4-5-3 and a guide rail 4-5-4. The gear 4-5-2 and the rack 4-5-3 can use plastic teeth with a height of no more than 5 mm. A base band of the rack 4-5-3 can be made of silicone, prefer to medical grade silicone, with a hardness degree to 60 degree, the rack 4-5-3 can be with a length of more than 40 cm, a thickness of more than 8 mm, and a width of more than 8 mm, and a upper end 4-5-3-1A and a lower end 4-5-3-1B of the rack 4-5-3 can be respectively provided with an upper pressure sensor 4-5-3-2A and a lower pressure sensor 4-5-3-2B. The guide rail 4-5-4 can be a semi-circular groove, a plurality of miniature rollers 4-5-4-1 can be arranged in the semi-circular groove, a upper block 4-5-4-2A and a lower block 4-5-4-2B can be arranged at ends of the guide rail 4-5-4 respectively, and the guide rail 4-5-4 can surround the gear 4-5-2 and the rack 4-5-3. The Cartesian coordinate robot can be based on an algorithm, and travel to a corresponding coordinate address. When the rack 4-5-3 moves with the gear 4-5-2, the upper end 4-5-3-1A or the lower end 4-5-3-1B of the guide rail 4-5-4 can protrude in a tangential direction, precisely insert into the first opening 3-4-1 of a specific medicine box 3-4, advance a certain distance, and push out a medicine in the medicine box 3-4, so that the medicine can pass through the second opening 3-4-2, and fall into the conveyor belt 5. When multiple medicines need to be pushed out, the Cartesian coordinate robot can plan the travel route according to a shortest running distance algorithm, then the upper end 4-5-3-1A and the lower end 4-5-3-1B of the guide rail 4-5-4 can extend and insert into the medicine box 3-4 with a bidirectional rotation of the gear 4-5-2. According to the algorithm, the medicine cabinet 3-1 can be equally divided into a upper medicine box 3-4 and a lower medicine box 3-4, when the medicine to be pushed out is located in the upper medicine box 3-4, the gear 4-5-2 rotates so that the upper end 4-5-3-1A of the rack 4-5-3 extends, and when the medicine to be pushed out is located in the lower medicine box 3-4, the gear 4-5-2 rotates so that the lower end 4-5-3-1B of the rack 4-5-3 extends. The guide rail 4-5-4 can includes a upper stopper 4-5-4-2A and a lower stopper 4-5-4-2B configured to prevent the rack 4-5-3 from disengaging from the gear 4-5-2. When multiple medicines need to be pushed out, a total of shortest distance running algorithm can plan the travel route of the coordinate robot and the conveyor belt 5 intelligent elevator. In addition, the Cartesian coordinate robot and its end effector 4-5 have a positioning accuracy of less than 0.5 mm.

Referring to FIG. 2, according to the embodiment of the present invention, a conveyor belt 5 can be arranged in the gap 3-3 at a certain distance according to a height of the drug store 3 to ensure that a drop height of a medicine does not exceed 50 cm. The conveyor belt 5 comprises a driving wheel, a driven wheel, a crawler, a roller, a roller, a bracket, a second driving motor, a first photoelectric sensor, and a controller. The driving wheels, the driven wheels, the tracks, the rollers, the rollers and the brackets can be made of soft foamed plastics, which can reduce a damage of medicines caused by falling and collision. The first photoelectric sensor is configured to monitor a quantity of the medicine arriving on the conveyor belt 5. After comparing with the quantity of medicine stated in a prescription, the quantity of medicine dispensed is reached, and the second drive motor works. In order to further reduce the damage caused by falling and collision, liquid medicines, medicines in glass bottles, and medicines with a weight of more than 100 g can be selected and distributed to the medicine boxes 3-4 that are close to a top of the conveyor belt 5 for storage.

In the embodiment of the present invention, the conveyor belt 5 can connected to an intelligently control elevator so that when the Cartesian coordinate robot end effector 4-5 pushes out the medicine, the intelligently control elevator moves synchronously so that the medicine leaves the medicine box 3-4 and falls on the conveyor belt 5, so as to reduce an impact of the medicine. When multiple medicines need to be pushed, the Cartesian coordinate robot and the intelligently control elevator can plan a travel route according to an algorithm with the shortest running distance. The intelligently control elevator can choose to use a screw system or a pulley block system.

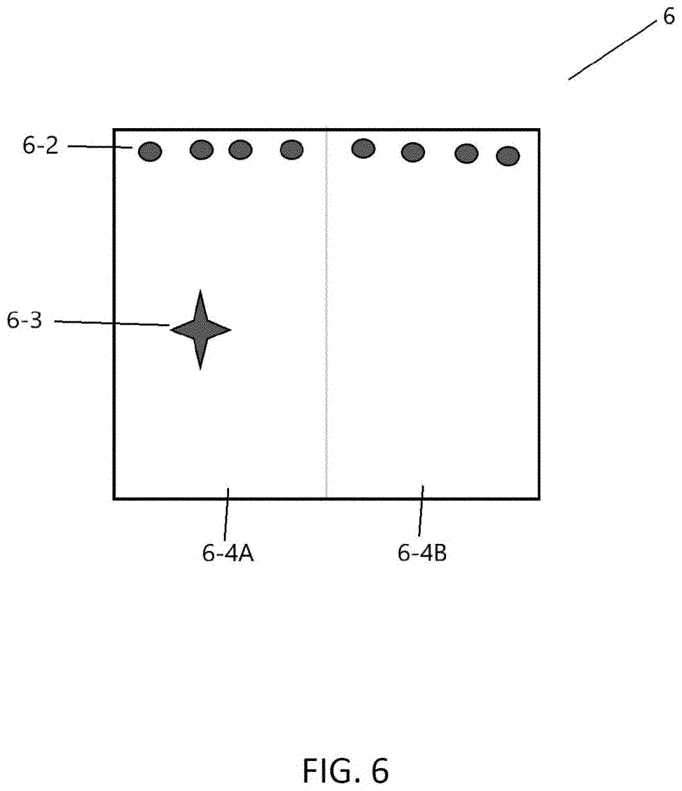

Referring to FIGS. 2 and 6, the induction door 6 according to the embodiment of the present invention can include a temporary storage concave plate 6-1, an automatic door 6-4, a second photoelectric sensor 6-5, a second light strip 6-5 composed of a diode lamp, and a second indicator light 6-3. The automatic door 6-4 use a left door 6-4A and a right door 6-4B to open in opposite directions. The number of induction doors 6 can be the same as the number of conveyor belts 5. The conveyor belt 5 can transport the medicine to the temporary storage concave tray 6-1, the second photoelectric sensor 6-5 can collect an information on the number of the medicine in the temporary storage concave tray 6-1, and the number of lights on the second light belt 6-2 can be used to dynamically display an arrival at the temporary storage tray 6-1. If the number of the medicine in the concave plate 6-1 is the same as the number of the medicine expected to arrive, the second indicator light 6-3 will be on, indicating that the medicine is taken, the automatic door 6-4 is opened, the medicine is taken, the automatic door 6-4 is closed, and the second indicator light 6-3 is off. If the medicine is not completed and the automatic door 6-4 is closed, the second indicator light 6-3 will continue to light up, reminding the completion of taking medicine, and the second light with the number of lights 6-2 is on to remind the number of the medicine were not picked up.

Referring to FIG. 1, according to the embodiment of the present invention, when the medicine is applied for the first time, the visual sensor 9 can be configured to scan and identify a supervision code, barcode, or outer packaging of the medicine, and retrieve it by means of a central data storage processor, a cloud server, or a network, obtain the medicine category, specification, dosage form, production date and expiration date, automatically add the medicine data to a database of the central data storage processor, and distribute a medicine boxes 3-4 appropriately according to the medicine weight, shape, length, width and height of an outer packaging. If the medicine box 3-4 is no suitable, the staff will be prompted to adjust the length, width and height of the medicine box 3-4 to accommodate the category, and the updated database will be uploaded to the cloud server synchronously. When the medicine is replenished again, the visual sensor 9 scans and recognizes the supervision code, barcode, or outer packaging of the medicine, and the medicine data is synchronously mapped to the database and cloud server of the central data storage processor, and the first indicator light 3-4-4 of the medicine box 3-4 corresponding to the medicine is on, the first light strip 3-4-3 composed of diode lights displays the number of medicine shortages, and guides the replenishment personnel to implement replenishment operation. When a patient sees a doctor, the visual sensor 9 can be configured to scan and identify the patient identity, identification card, medical insurance card, and credit card, scan the patient face and body features, and assist in diagnosis.

According to the embodiment of the present invention, a body temperature detection sensor, a pulse detection sensor, a breathing motion sensor, a blood pressure detection sensor, and a blood oxygen saturation detection sensor can be provided on a front side of the central shell 2-1, which can be used for a patient self-service physical examination with a help of a display 7 and a smart speaker 8. A built-in wireless communication equipment in the right edge shell 2-2B can include a near field communication component and a remote communication component, the near field communication component can be used for an internal communication of the device and the communication with a nearby patient's mobile intelligent terminal, the remote communication component can be used for data uploading and downloading, and remote diagnosis and treatment. The central data storage processor can be built in the right edge shell 2-2B, which includes a control unit, an arithmetic unit and a storage unit. The control unit can be configured for intelligent control of an internal controller of the device, such as the first light strip 3-4-3 diode light, the second light strip 6-2 diode lamp, the first indicator light 3-4-4, the second indicator light 6-3, the Cartesian coordinate robot and its end effector 4-5 and its servo motor 4-5-1, the first photoelectric sensor, a second photoelectric sensor 6-5, an elevator drive motor, and an automatic door 6-4. The arithmetic unit can include a CPU, a GPU, and an NPU, which are configured for an execution of an algorithm. The algorithm includes the medicine box 3-4 coordinate positioning (X, Y), the medicine coordinate positioning (X, Y, X), the medicine category identification and statistics, the Cartesian coordinate robot end effector 4-5 travel planning, sensor acquisition data processing, human-machine interaction data processing, and further processing of cloud server computing data. The storage unit includes an internal memory and an external memory for a storing system software, an application program, the algorithm, a temporary data and short-term storage of a permanent data.

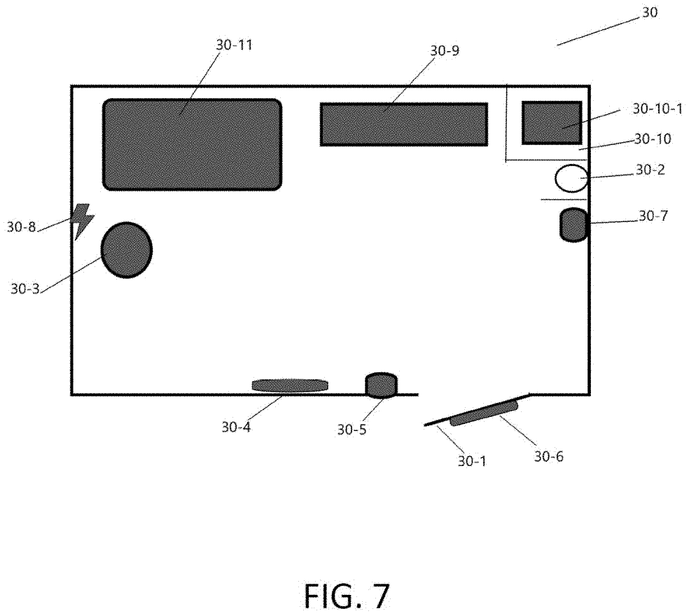

Referring to FIG. 7, a pharmacy kiosk 30 according to the embodiment of the present invention can use part of an existing building for isolation or use an independent building to make a closed or semi-enclosed space. The pharmacy kiosk 30 can be powered, water, ventilated, and 5G connected. The pharmacy kiosk 30 at least includes a door 30-1, a lock, a plurality of walls, ceilings and floors, a pharmacy robot placement area 30-11, a stool 30-3, a ramp, a lighting lamp 30-4, an audio and video camera 30-5, a second display 30-6, sound player 30-7, an emergency button 30-8, a disinfection equipment, and an air conditioner. The pharmacy kiosk 30 can accommodate at least a single pharmacy robot 10 or a placement area 30-11 of the pharmacy robot complex 20 and at least a wheelchair and a stool 30-3. The disinfection equipment includes an ultraviolet lamp and a disinfectant spray device.

Referring to FIG. 7, according to the embodiment of the present invention, the pharmacy kiosk 30 can also be provided with a hand washing station 30-2, a medical equipment table 30-9, and a toilet 30-10. The medical equipment table 30-9 can be provided with an intelligent stethoscope, an intelligent percussion hammer, a fully automatic electrocardiograph, a self-service respiratory function tester, a self-service EEG tester, a self-service finger blood collection equipment, a self-service blood sugar tester, an automatic blood routine analyzer. The toilet 30-10 can be equipped with a smart toilet 30-10-1 with an automatic urine and stool analyzer. It is convenient for a patient to perform auscultation, percussion, electrocardiogram, respiratory function, EEG examination, self-collection of finger blood for rapid blood sugar detection, and blood and urine analysis.

According to the embodiment of the present invention, the pharmacy kiosk 30 can be located in a remote town or a rural area or a nearby community lacking a hospital. The pharmacy kiosk 30 can be isolated and serve nearby residents. The pharmacy kiosk can be isolated at a self-service area inside or outside a pharmacy, as well as in an outpatient of a hospital. Of course, in public gathering places, such as commercial areas, stations, airports, and service halls of passenger ferry terminals, the pharmacy kiosks 30 can also be isolated to provide medicine and medical services.

According to the embodiment of the present invention, an air conditioner can be installed in the chassis 1 to adjust a temperature and humidity in the drug store 3, an ozone generator can be installed in the chassis 1, which can be connected to the drug store 3 through a pipeline, so that air inside the drug store 3 can be sterilized. In addition, the chassis 1 can be provided with a disinfectant storage tank, a spray equipment and a plurality of pipelines, the pipelines are respectively opened to the temporary storage concave plate 6-1, the display 7 and the shell 2.

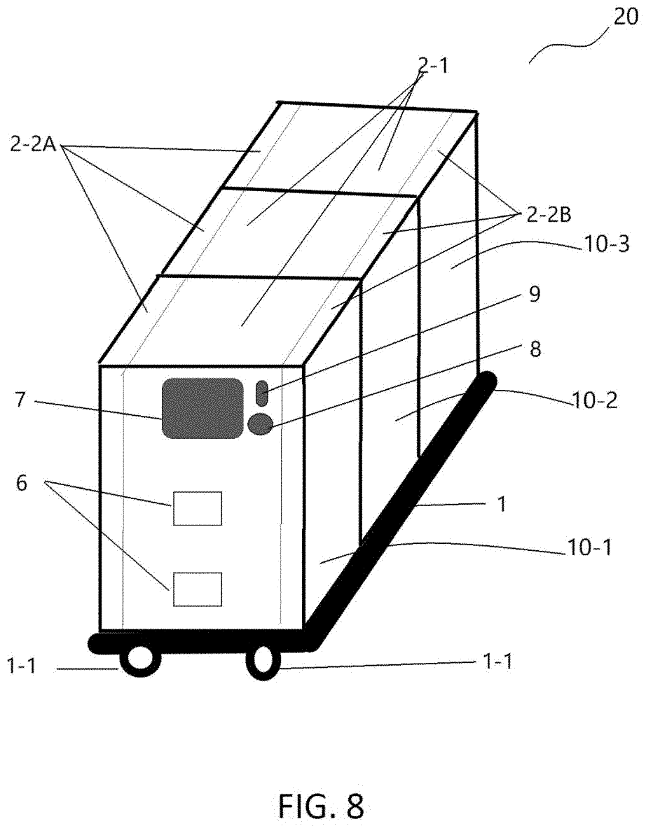

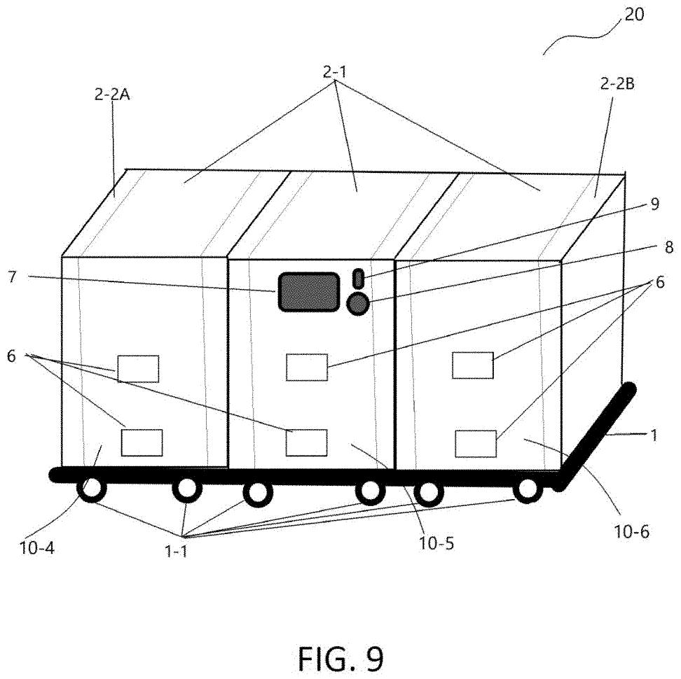

As shown in FIGS. 8 and 9, a method for connecting a pharmacy robot device according to the embodiment of the present invention can include a single pharmacy robot 10 in series, a single pharmacy robot 10 in parallel, or a single pharmacy robot 10 in series and parallel. It is necessary to combine an actual need and comprehensive planning of a structure of a pharmacy kiosk.

Referring to FIG. 8, one embodiment of the present invention takes three single pharmacy robots 10 connected in series as an example to illustrate that the conjoined combination becomes a pharmacy robot complex 20. In practical applications, two, four or more single pharmacy robots 10 can be used in series. A front single pharmacy robot 10-1, a middle single pharmacy robot 10-2, and a rear single pharmacy robot 10-3 are connected in series in a front-to-rear arrangement. A chassis 1, a drug store 3, a left edge shell 2-2A, a right edge shell 2-2B, a push rod 4, and a conveyor belt 5 of the three pharmacy robots are kept independent, and an induction door 6, a display 7, a smart speaker 8, a visual sensor 9, and a central data storage processor are shared. The conveyor belt 5, an electric circuit, and a communication line are connected with each other. A plurality of openings on the front side of a center shell 2-1 rear side of the front single pharmacy robot 10-1, a center shell 2-1 front and rear sides of the middle single pharmacy robot 10-2, and a center shell of the rear single pharmacy robot 10-3 2-1 can be set at a position corresponding to the induction door 6, so that the conveyor belts 5 of the three single pharmacy robots are seamlessly connected, and the medicine pushed out from a medicine box 3-4 are transported to the shared temporary storage concave tray 6-1, thereby greatly increasing the medicine categories and quantities stored in the drug store 3.

Referring to FIG. 9, one embodiment of the present invention takes three single pharmacy robots 10 in parallel as an example to illustrate that the conjoined combination becomes a pharmacy robot complex 20. In practical applications, different numbers of single pharmacy robot 10 can be used in parallel. A left single pharmacy robot 10-4, a center single pharmacy robot 10-5, and a right single pharmacy robot 10-6 are connected in parallel in a left-right arrangement. A center shell 2-1, a left edge shell 2-2A, a right edge shell 2-2B, an induction door 6, a push rod 4, and a conveyor belt 5 remain independent settings, a display 7, a smart speaker 8, a vision sensor 9, and a center data storage processor are shared, and a circuit and a communication line are connected with each other, configured to increase the medicine categories and quantities stored in the drug store 3. In addition, a plurality of single pharmacy robots 10 can be arranged in series and parallel to form a pharmacy robot complex 20 with the display 7, the smart speaker 8, the visual sensor 9, and the central data storage processor shared, and the circuits and communication lines connected, configured to increase categories and quantities of medicines and medical equipment.

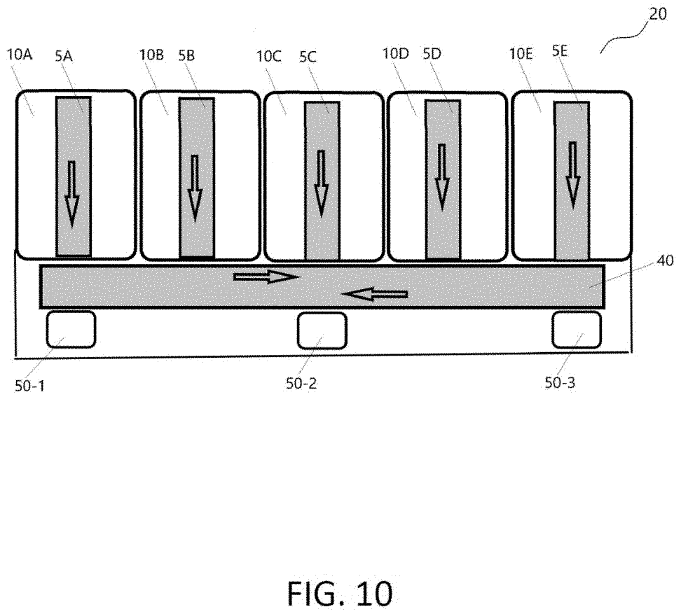

As shown in FIGS. 10 to 20, one embodiment of the present invention takes five single pharmacy robots 10 in parallel or series-parallel and three medicine outlets as an example to describe a device and method for medicine delivery in a pharmacy robot complex 20.

Referring to FIG. 10, a pharmacy robot complex 20 according to the embodiment of the present invention can comprise a first single pharmacy robot 10A, a second single pharmacy robot 10B, a third single pharmacy robot 10C, a fourth single pharmacy robot 10D, a fifth single pharmacy robot 10E, a crawler carrying device 40, a first collection bin 50-1, a second collection bin 50-2, and a third collection bin 50-3. A medicine of the first single pharmacy robot 10A, the second single pharmacy robot 10B, the third single pharmacy robot 10C, the fourth single pharmacy robot 10D and the fifth single pharmacy robot 10E can pass through the first conveyor belt 5A, the second conveyor belt. 5B, the third conveyor belt 5C, the fourth conveyor belt 5D, and/or the fifth conveyor belt 5E, be transferred to the crawler carrying device 40. According to an instruction, the crawler carrying device 40 can reciprocate to transport the medicine to an area of the first collecting bin 50-1 or the second collecting bin 50-2 or the third collection bin 50-3, a visual sensor identifies the medicine, a pushing device pushes the medicine into the first collection bin 50-1, or the second collection bin 50-2, or the third collection bin 50-3.

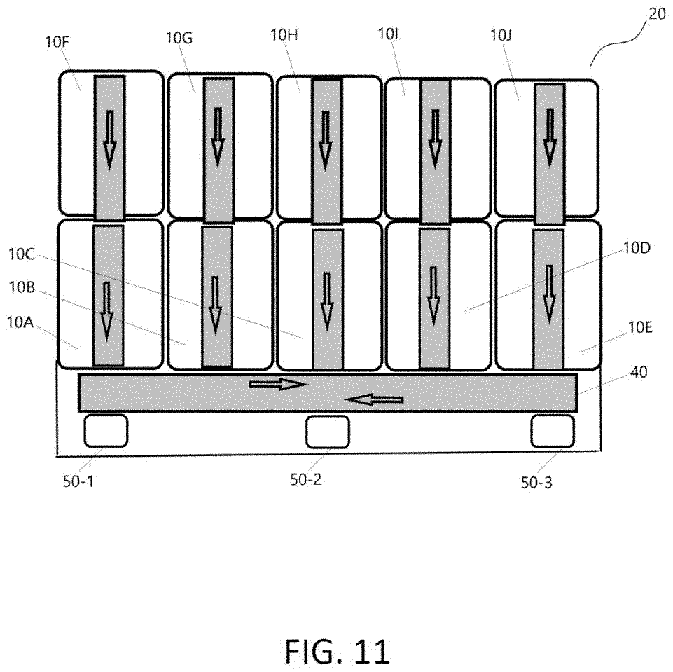

As shown in FIG. 11, a pharmacy robot complex 20 according to the embodiment of the present invention can comprise a first single pharmacy robot 10A, a second single pharmacy robot 10B, a third single pharmacy robot 10C, a fourth single pharmacy robot 10D and the fifth single pharmacy robot 10E in parallel, and a sixth single pharmacy robot 10F, a seventh single pharmacy robot 10G, an eighth single pharmacy robot 10H, a ninth single pharmacy robot 10I and a tenth single pharmacy robot 10J in series and parallel, a crawler carrying device 40, a first collecting bin 50-1, a second collecting bin 50-2, and a third collecting bin 50-3. The conveyor belts 5 of the first single pharmacy robot 10A, the second single pharmacy robot 10B, the third single pharmacy robot 10C, the fourth single pharmacy robot 10D, and the fifth single pharmacy robot 10E are respectively connected with the sixth single pharmacy robot 10F, the seventh single pharmacy robot 10G, the eighth single pharmacy robot 10H, the ninth single pharmacy robot 10I, and the tenth single pharmacy robot 10J to realize the medicine of the sixth single pharmacy robot 10F, the seventh single pharmacy robot 10G, the eighth single pharmacy robot 10H, the ninth single pharmacy robot 10I, and the tenth single pharmacy robot 10J pass through the conveyor belts 5 of the first single pharmacy robot 10A, the second single pharmacy robot 10B, the third single pharmacy robot 10C, the fourth single pharmacy robot 10D, and the fifth single pharmacy robot 10E, and transport to the crawler carrying device 40. According to an instruction, the medicine can be pushed into the first collection bin 50-1, or the second collection bin 50-2, or the third collection bin 50-3.

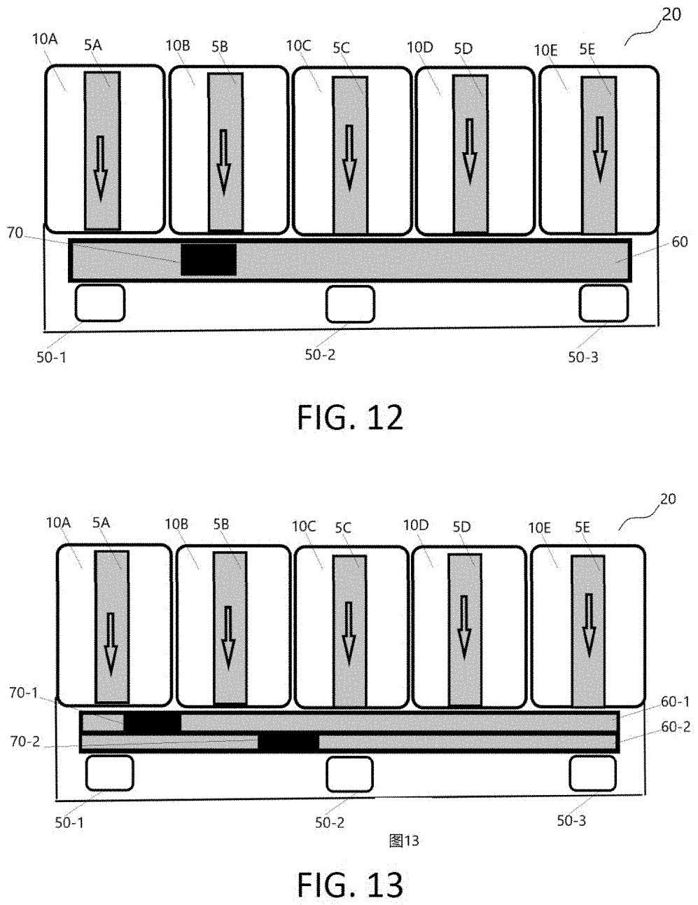

Referring to FIG. 12, a pharmacy robot complex 20 according to the embodiment of the present invention can comprise a first single pharmacy robot 10A, a second single pharmacy robot 10B, a third single pharmacy robot 10C, a fourth single 1 pharmacy robot 10D, a fifth single pharmacy robot 10E, a single track 60, an unmanned vehicle 70, a first collection bin 50-1, a second collection bin 50-2, and a third collection bin 50-3. According to an instruction, the unmanned vehicle 70 can reciprocate on the single track 60. The medicine of the first single pharmacy robot 10A, the second single pharmacy robot 10B, the third single pharmacy robot 10C, the fourth single pharmacy robot 10D, and the fifth the single pharmacy robot 10E can be delivered to the unmanned vehicle 70, and the unmanned vehicle 70 transports the medicine to a corresponding area of the first collection bin 50-1, the second collection bin 50-2, or the third collection bin 50-3, the visual sensor recognizes the medicine, and the pushing device pushes the medicine into the first collection bin 50-1, the second collection bin 50-2, or the third collection bin 50-3.

As shown in FIG. 13, a pharmacy robot complex 50 according to the embodiment of the present invention can comprise a first single pharmacy robot 10A, a second single pharmacy robot 10B, a third single pharmacy robot 10C, a fourth single pharmacy robot 10D, a fifth single pharmacy robot 10E, a first track 60-1, a second track 60-2, a first unmanned vehicle 70-1, a second unmanned vehicle 70-2, a first collection bin 50-1, a second collection bin 50-2 and a third collection bin 50-3. According to an instruction, the first unmanned vehicle 70-1 and the second unmanned vehicle 70-2 can reciprocate on the first track 60-1 and the second track 60-2 respectively. The medicine of the first single pharmacy robot 10A, the second single pharmacy robot 10B, the third single pharmacy robot 10C, the fourth single pharmacy robot 10D, and the fifth single pharmacy robot 10E can be delivered to the first unmanned vehicle 70-1 and/or the second unmanned vehicle 70-2, the first unmanned vehicle 70-1 and/or the second unmanned vehicle 70-2 deliver the medicine to a corresponding area of the first collection bin 50-1, or the second collection bin 50-2, or the third collection bin 50-3, a visual sensor recognizes the medicine, then a pushing device pushes the medicine into the first collection bin 50-1, the second collection bin 50-2, or the third collection bin 50-3.

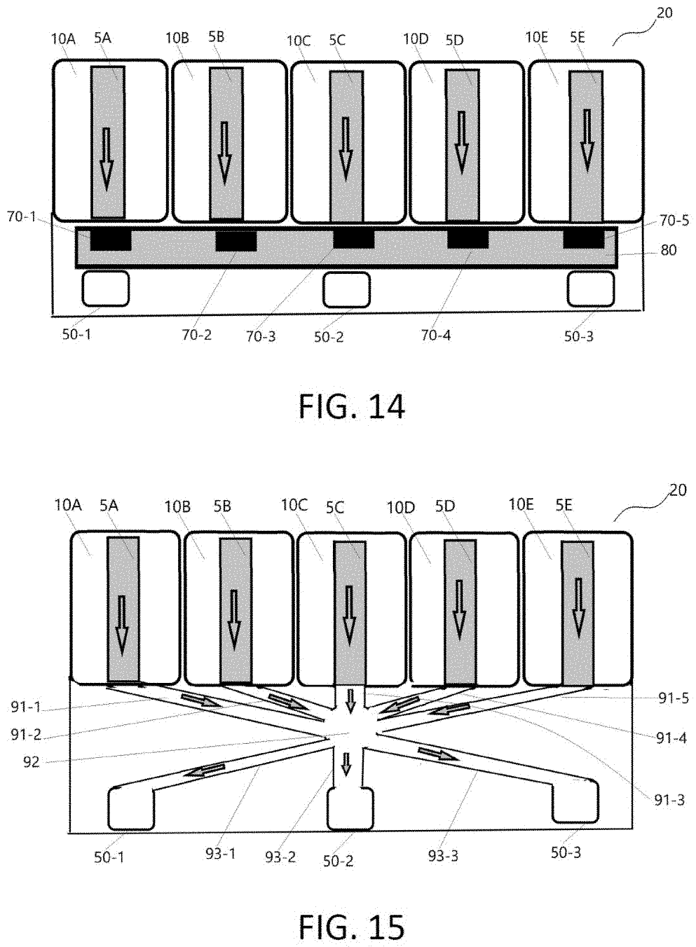

As shown in FIG. 14, a pharmacy robot complex 20 according to the embodiment of the present invention can comprise a first single pharmacy robot 10A, a second single pharmacy robot 10B, a third single pharmacy robot 10C, a fourth single pharmacy robot 10D, a fifth single pharmacy robot 10E, a road platform 80, a first unmanned vehicle 70-1, a second unmanned vehicle 70-2, a third unmanned vehicle 70-3, a fourth unmanned vehicle 70-4, a fifth unmanned vehicle 70-5, a first collection bin 50-1, a second collection bin 50-2, and a third collection bin 50-3. According to an instruction, the first unmanned vehicle 70-1, the second unmanned vehicle 70-2, the third unmanned vehicle 70-3, the fourth unmanned vehicle 70-4, and the fifth unmanned vehicle 70-5 can be on standby or running on the road platform 80, transport a medicine to a corresponding area of the first collection bin 50-1, the second collection bin 50-2, or the third collection bin 50-3, a visual sensor recognizes the medicine, and a push device pushes the medicine into the first collection bin 50-2. or the second collecting bin 50-2, or the third collecting bin 50-3.

Referring to FIG. 15, a pharmacy robot complex 20 according to the embodiment of the present invention can comprise a first single pharmacy robot 10A, a second single pharmacy robot 10B, a third single pharmacy robot 10C, a fourth single pharmacy robot 10D, a fifth single pharmacy robot 10E, a first proximal slide 91-1, a second proximal slide 91-2, a third proximal slide 91-3, a fourth proximal slide 91-4, a fifth proximal slide 91-5, a central slide way 92, a first distal slide way 93-1, a second distal slide way 93-2, a third distal slide way 93-3, a first collection bin 50-1, a second collection bin 50-2 and a third collection bin 50-3.

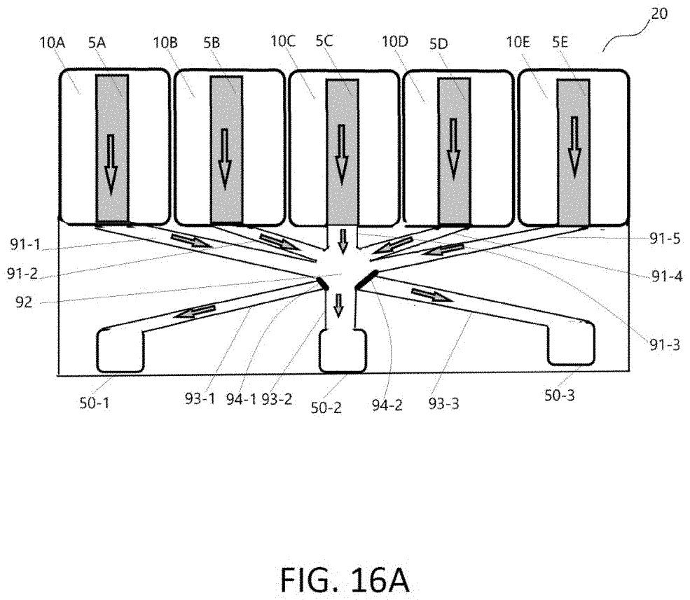

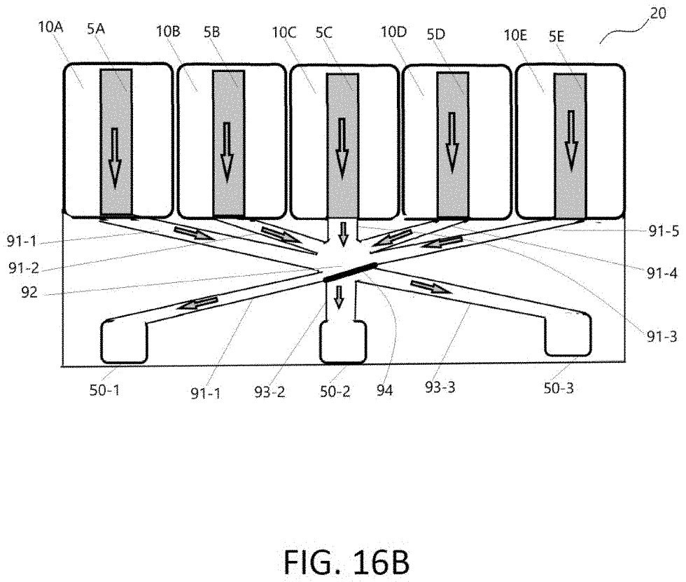

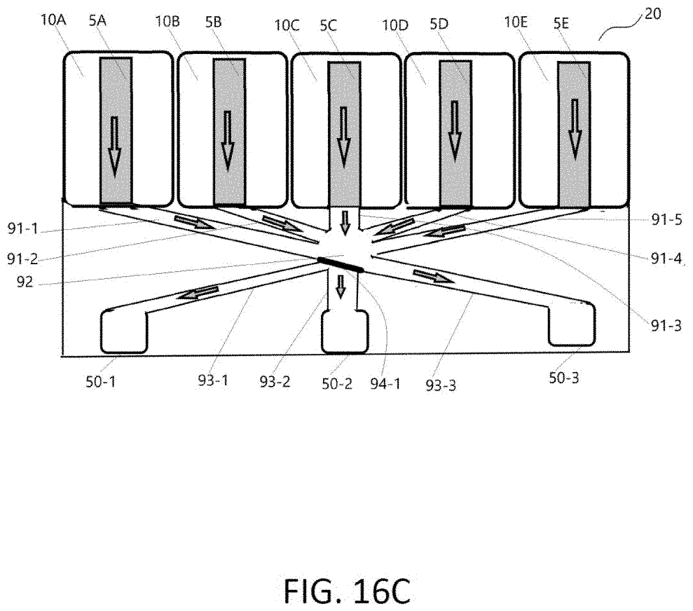

As shown in FIG. 16A, in a pharmacy robot complex 20 according to the embodiment of the present invention, a robot arm can place a switch 94 into a central slide way 92 according to an instruction, a first switch 94-1 and a second switch 94-2 respectively block a first distal slide way 93-1 and a third distal slide way 93-3, so as to make a first proximal slide 91-1, a second proximal slide 91-2, a third proximal slide 91-3, a fourth proximal slide 91-4, and a fifth proximal slide 91-5 connected to a second collection bin 50-2 through the central slide way 92 and a second distal slide way 93-2, the medicine of a first single pharmacy robot 10A, a second single pharmacy robot 10B, a third single pharmacy robot 10C, a fourth single pharmacy robot 10D, and a fifth single pharmacy robot 10E can enter the second collection bin 50-2.

As shown in FIG. 16B, in a pharmacy robot complex 20 according to the embodiment of the present invention, according to an instruction, a robot arm can place a switch 94 into a central slide 92, a first switch 94-1 blocks a second distal slide 93-2 and a third distal slide 93-3, so that a first proximal slide 91-1, a second proximal slide 91-2, a third proximal slide 91-3, a fourth proximal slide 91-4, and a fifth proximal slide way 91-5 can be connected to a first collection bin 50-1 through the central slide way 92 and a first distal slide way 93-1, a medicine of a first single pharmacy robot 10A, a second single pharmacy robot 10B, a third single pharmacy robot 10C, a fourth single pharmacy robot 10D, and a fifth single pharmacy robot 10E can enter the first collection bin 50-1.

As shown in FIG. 16C, in a pharmacy robot complex 2 according to the embodiment of the present invention, according to an instruction, a robot arm can place a switch 94 into a central slide 92, a first switch 94-1 blocks a first distal slide 93-1 and a second distal slide 93-2, so that a first proximal slide 91-1, a second proximal slide 91-2, a third proximal slide 91-3, a fourth proximal slide 91-4 and a fifth proximal slide way 91-5 can be connected to a third collection bin 50-3 through the central slide way 92 and a third distal slide way 93-3, a medicine of a first single pharmacy robot 10A, a second single pharmacy robot 10B, a third single pharmacy robot 10C, a fourth single pharmacy robot 10D, and a fifth single pharmacy robot 10E can enter the third collection bin 50-3.

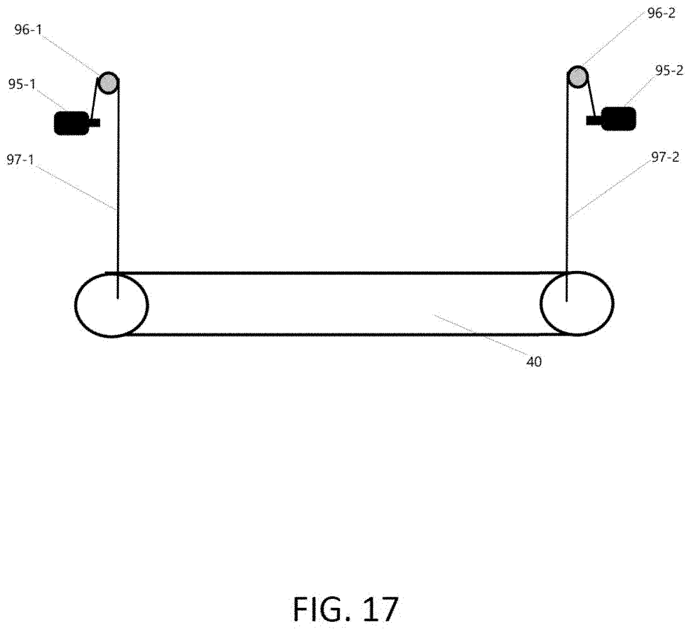

Referring to FIG. 17, a pharmacy robot complex 20 according to the embodiment of the present invention can comprise a crawler carrying device 40, a first smart motor 95-1, a first pulley 96-1, a first rope 97-1, a second smart motor 95-2, a second pulley 96-2, and a second rope 97-2, which become a pulley lifting device. One end of the first rope 97-1 and the second rope 97-2 can be around a rotating shaft of the first intelligent motor 95-1 and the second intelligent motor 95-2 respectively, the other end of the first rope 97-1 and the second rope 97-2 are respectively fixed to the crawler carrying device 40. According to an instruction, the first intelligent motor 95-1 and the second intelligent motor 95-2 run synchronously, the crawler carrying device 40 can be accurately raised or lowered by winding or releasing the first rope 97-1 and the second rope 97-2.

Of course, the pulley lifting device of the pharmacy robot complex 20 according to the embodiment of the present invention can also be combined with a single track 60, a first track 60-1, a second track 60-2, a road platform 80, a first collection bin 50-1, a second collection bin 50-2, and a third collecting bin 50-3, so that the single track 60, the first track 60-1, the second track 60-2, the road platform 80, the first collecting bin 50-1, the second collecting bin 50-2 and the third collecting bin 50-3 can be raised or lowered.

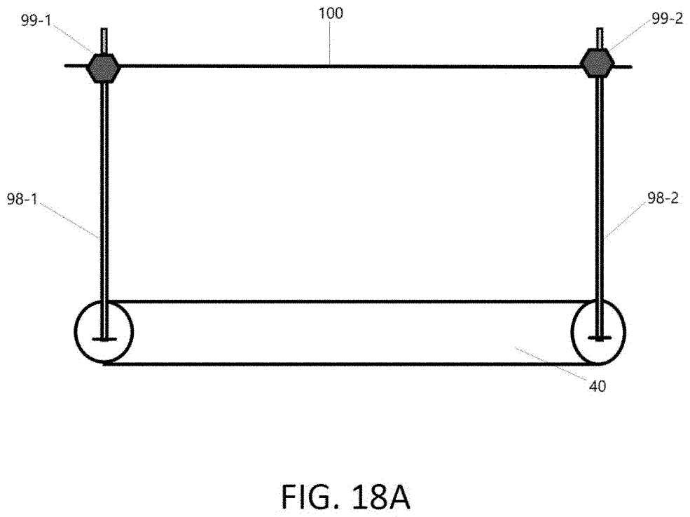

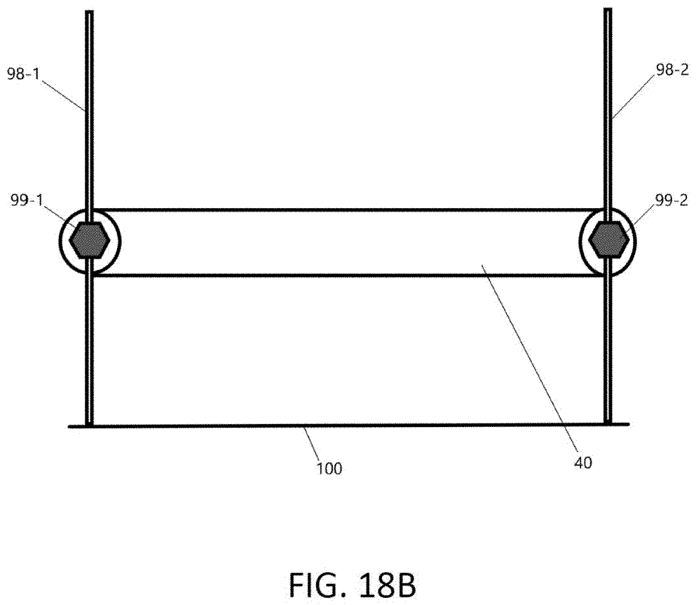

Referring to FIG. 18A, a pharmacy robot complex 20 according to the embodiment of the present invention can further comprise a crawler carrying device 40, a first vertical rod 98-1, a first intelligent motor nut complex 99-1, a fixing frame 100, and a second vertical rod 98-2. The second intelligent motor nut complex 99-2, the first intelligent motor nut complex 99-1, and the second intelligent motor nut complex 99-2 can be respectively connected to the fixing frame 100. One ends of the first vertical rod 98-1 and the second vertical rod 98-2 can be respectively fixed to two ends of the crawler carrying device 40, and the other ends of the first vertical rod 98-1 and the second vertical rod 98-2 can be free. According to an instruction, the first intelligent motor nut complex 99-1 and the second intelligent motor nut complex 99-2 run synchronously, the crawler carrying device 40 can be driven to up or lower by the first vertical rod 98-1 and the second vertical rod 98-2.

Of course, as shown in FIG. 18A, a screw axial movement screw lifting device composed of the second intelligent motor nut complex 99-2, the first vertical rod 98-1, the first intelligent motor nut complex 99-1, the fixing frame 100, and the second vertical rod 98-2 of the pharmacy robot complex 20 according to the embodiment of the present invention can also be combined with a single track 60, a first track 60-1, a second track 60-2, a road platform 80, a first track, a first collecting bin 50-1, the second collecting bin 50-2, and a third collecting bin 50-3, so that the single track 60, the first track 60-1, the second track 60-2, the road platform 80, the first collecting bin 50-1, the second collecting bin 50-2 and/or the third collecting bin 50-3 can be raised or lowered.

Referring to FIG. 18B, a pharmacy robot complex 20 according to the embodiment of the present invention further comprises a crawler carrying device 40, a first vertical rod 98-1, a first intelligent motor nut complex 99-1, a fixing frame 100, and a second vertical rod 98-2. The second intelligent motor nut complex 99-2, the first intelligent motor nut complex 99-1 and the second intelligent motor nut complex 99-2 are respectively fixed to both ends of the crawler carrying device 40. One ends of the first vertical rod 98-1 and the second vertical rod 98-2 are respectively fixed to the fixing frame 100, and the other ends of the first vertical rod 98-1 and the second vertical rod 98-2 are free. According to an instruction, the first intelligent motor nut complex 99-1 and the second intelligent motor nut complex 99-2 can run synchronously, and the first intelligent motor nut complex 99-1 and the second intelligent motor nut complex 99-2 drive the crawler carrying device 40 up or lower.

Of course, as shown in FIG. 18B, a nut axial movement screw lifting device composed of the second intelligent motor nut complex 99-2, the first vertical rod 98-1, the first intelligent motor nut complex 99-1, the fixing frame 100, and the second vertical rod 98-2 of the pharmacy robot complex 20 according to the embodiment of the present invention can also be combined with a single track 60, a first track 60-1, a second track 60-2, a road platform 80, a first collection bin 50-1, a second collection bin 50-2, and a third collection bin 50-3, so that the single track 60, the first track 60-1, the second track 60-2, the road platform 80, the first collection bin 50-1, the second collection bin 50-2 and/or the third collection bin 50-3 can be raised or lowered.

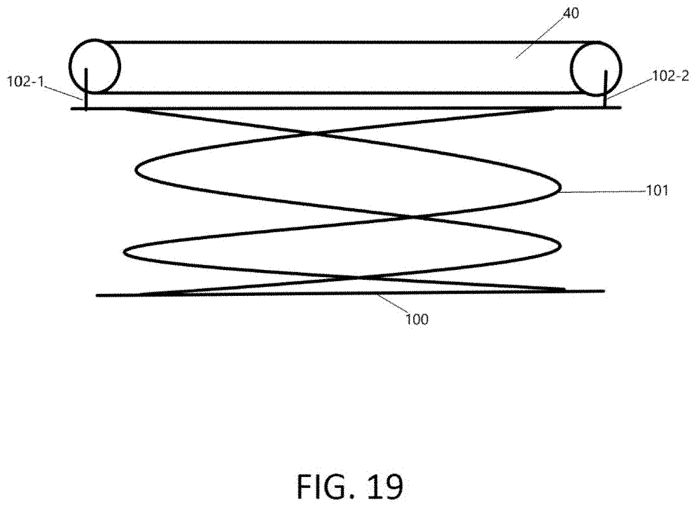

Referring to FIG. 19, a pharmacy robot complex 20 according to the embodiment of the present invention can further comprise a crawler carrying device 40, a hydraulic scissor lift platform device 101, a fixing frame 100, a first fixing seat 102-1, and a second fixing seat 102-2, The lower end of the hydraulic scissor lift platform device 101 is connected to the fixing frame 100, and the upper end is connected to the crawler carrying device 40 through the first fixing seat 102-1 and the second fixing seat 102-2 respectively. The hydraulic scissor lifting platform device 101 can drive the crawler carrying device 40 accurately raise or lower.

Of course, a scissor lift platform system composed of the hydraulic scissor lift platform device 101, the fixing frame 100, the first fixing seat 102-1 and the second fixing seat 102-2 of a pharmacy robot complex 20 as shown in FIG. 19 can also be combined with a single track 60, a first track 60-1, a second track 60-2, a road platform 80, a first collection bin 50-1, a second collection bin 50-2, and a third collection bin 50-3, so that the single track 60, the first track 60-1, the second track 60-2, the road platform 80, the first collection bin 50-1, the second collection bin 50-2, and/or the third collection bin 50-3 can be raised or lowered.

In addition, a hydraulic scissor lift platform of the pharmacy robot complex 20 according to the embodiment of the present invention as shown in FIG. 19 can also be replaced by a single-mast hydraulic lift platform or a double-mast hydraulic lift platform for a crawler carrying device 40, a single track 60, a first rail 60-1, a second rail 60-2, a road platform 80, a first collection bin 50-1, a second collection bin 50-2, and/or a third collection bin 50-3 raised or lowered.

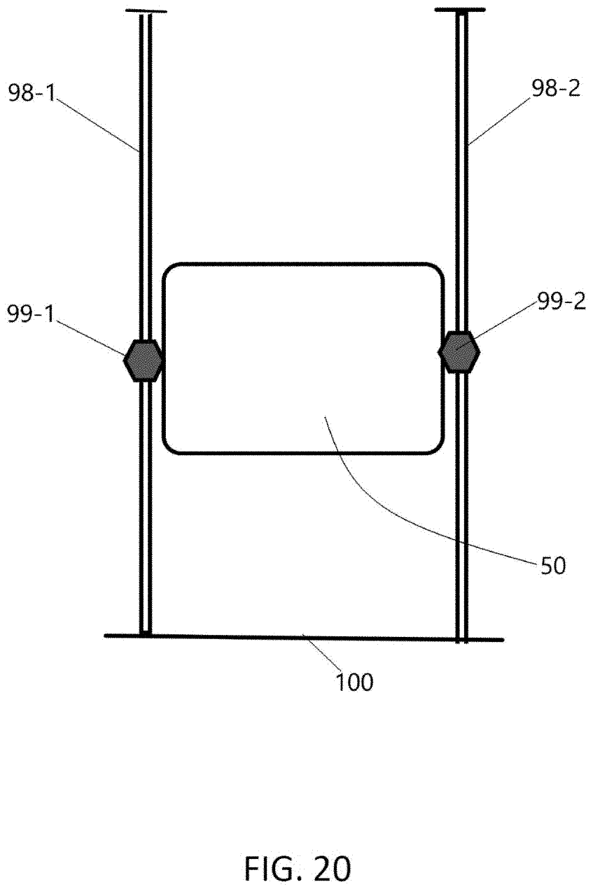

Referring to FIG. 20, a pharmacy robot complex according to the embodiment of the present invention can comprise a collection bin 50, a first vertical rod 98-1, a first intelligent motor nut complex 99-1, a fixing frame 100, a second vertical rod 98-2, and a second intelligent motor nut complex 99-2, the first intelligent motor nut complex 99-1 and the second intelligent motor nut complex 99-2 are fixed to the collection bin 50, one end of the first vertical rod 98-1 and the second vertical rod 98-2 is respectively fixed to the fixing frame 100, and the other ends of the first vertical rod 98-1 and the second vertical rod 98-2 are free. According to an instruction, the first intelligent motor nut complex 99-1 and the second intelligent motor nut complex 99-2 run synchronously, driving the collection bin 50 to rise or fall.

A method process of a pharmacy robot device according to the embodiment of the present invention can comprise a drug sale process, a supplementation process, a drug inventory process, a connection process, and a grid deployment process.

A drug sale process 200 according to the embodiment of the present invention can comprise a plurality of steps as follows:

210: Pharmacy is a doctor service, which can include a plurality of steps as follow:

212: A patient uses a visual sensor to scan on biometrics, passes identity authentication, and uses a smart speaker or a display for an interaction, then a main appeals and symptoms can be submitted through the interaction, the visual sensor collects the patient face and tongue image data, an intelligent diagnosis and treatment system judges whether there is an acute or chronic disease face, nutritional abnormality, emotional abnormality, or mental abnormality, and supplements a medical history, and then a recent paper or electronic version of a medical examination report, and/or prompting examination report be scanned and recognized by the visual sensor or uploaded through the patient mobile smart terminal App, combined with a real-time detection of basic physical signs such as body temperature, pulse, respiration, blood pressure, blood oxygen saturation and other data and a result of a rapid blood glucose and blood test, with an authorization of the patient, calla the patient previous health data, medication history, and allergy history, and then the intelligent diagnosis and treatment system infers a preliminary clinical diagnosis, issues a treatment recommendation, and issues an electronic prescription, the patient is reminded to go to a hospital for treatment, and the patient authorizes a smart contract signed by the patient to reflect his or her wish, whose past health data, medication history, and allergy history data are stored in a public cloud server or private industry server using block chain encryption technology.

214: The patient remotely interacts with voice or video through the mobile smart terminal App, submits a main appeal and symptom, uploads a face information, a tongue information, a voice information, a body temperature, a pulse, a respiration, a blood pressure collected by the mobile smart terminal, a blood oxygen saturation and other basic sign data, if there is a recent paper or electronic version of a medical examination report, upload it through the mobile smart terminal App, with an authorization of the patient, calls previous health data, medication history, allergy history, supplementary medical history, an intelligent diagnosis and treatment system infers a preliminary diagnosis, issues treatment recommendations, issues electronic prescriptions, and reminds the patient to go to a hospital if necessary.

216: When purchasing an over-the-counter drug, the patient directly submits an over-the-counter drug purchase requirement with a human-machine interaction through a smart speaker or a display.

218: If the patient directly purchases a prescription drug, the patient needs to provide a paper or electronic prescription issued by a doctor; if not, enter an on-site process of the step 212.

220: Pharmacy is a pharmacist service, which can include a plurality of steps as follow:

222: The electronic prescription obtained by the patient in the steps 212 and 214, or the electronic prescription imported through the mobile smart terminal App, is uploaded and stored in the cloud server, and the face, social security card, or ID card is recognized, and the name, gender, Age, height, weight, contact information, allergy history, disease conditions, medical diagnosis, check the medicine and dosage, check the doctor's signature, and ensure that the prescription is complete, accurate and true. Whether there is a shortage or excess, and whether there is a drug incompatibility, among which, checking the physician's signature is achieved by calling the database physician's qualification data, electronic signature or electronic seal record data.

224: The patient scans or takes pictures of the paper prescription and imports it through the mobile smart terminal App, or places the paper prescription in the vision sensor scanning area of the pharmacy robot for data collection and identification of key prescription information. Face recognition combined with social security card or ID card Identify, check the prescription, check the diagnosis, check the medicine, check the dosage, check the doctor's signature to ensure the completeness, accuracy and authenticity of the prescription. After encrypted by the block chain technology and stored in the cloud server, the party will be stored in the database. The callout has been used.

226: When a patient buys an over-the-counter drug, they only need face recognition combined with social security card scanning to check the patient. Through human-computer interaction, the patient can directly select the type and quantity of the drug.

228: There is no medicine in the single pharmacy robot or the pharmacy robot complex, the treatment method is as follows:

228-1: The patient can choose the drug arrival notification service through human-computer interaction, or

228-2: accept the prompt information and go to the nearby outlet or pharmacy where the stock is in stock, or

228-3: choose the third-party logistics for drug delivery door-to-door service.

230: The patient confirms whether to purchase the medicine through human-computer interaction, if so, enter 240, if not, the service is terminated.

240: The patient pays the fee.

250: The Cartesian coordinate robot addresses the position of the medicine box where the medicine is located, the end effector rack of the push rod device extends into the corresponding medicine box, pushes out the medicine, the medicine falls into the conveyor belt, and the conveyor belt sends the medicine to the temporary storage concave tray. After all the medicines have entered the temporary storage concave tray, the second indicator light of the induction door is on, and at the same time, the number of lights on the second row of diode lights is the same as the number of all medicines. After the sensor or the down pressure sensor senses the drug packaging box, each time the length (cm) of one drug packaging box is advanced, a box of drugs is pushed out until the required number of drug boxes is reached.

260: The left door and the right door of the induction door are opened opposite to each other, the patient takes medicine, the second photoelectric sensor monitors the temporary storage concave plate, and judges whether all the medicines are taken away, if not, the second indicator light continues to light, and the diode light. The number of lights on in the second light row is the same as the number of medicines not taken from the temporary storage concave tray, and is prompted by voice interaction of the smart speaker or the touch screen display. If yes, go to 300.

A pharmacy as a pharmacy management service process 300 according to the embodiment of the present invention can comprise a plurality of steps as follows.

310: A drug supply chain service, which includes a data sharing with a drug manufacturer, and enabling the manufacturer to sense changes in market demand so as to adjust drug production capacity in a timely manner, and clarify research and development directions, and a third-party supply chain dynamically sense drug inventories so as to provide drug replenishment through logistics, and/or replace drugs approaching expiration dates, and/or recall problem drug.

320: A pharmacy service can include a plurality of steps as follows:

322: Receive an imported electronic prescription, and send service information and electronic prescription to a patient mobile intelligent terminal.

324: The electronic prescription and/or paper prescription are marked on a cloud server industry-wide shared database, indicating that the prescription has been dispensed and taken, so that the prescription will not be used for repeated drug purchases.

326: Follow-up, including sending medication precautions, indications, contraindications, how to take medicines, etc. to the patient mobile smart terminal, timely reminding the patient to take the correct medication on time, and identifying and handling abnormal conditions after medication.

328: Chronic disease drugs are about to run out, remind the patient to purchase drugs in advance through the mobile smart terminal, and remind the patient to regularly check and evaluate treatment effects and life quality changes through the mobile smart terminal, and provide the patient with increased drug doses based on an inspection and evaluation result, or reduction recommendations, or recommendations for changing treatment regimens.

330: Pharmacy management, which includes a real-time statistics of drug store information, monitoring whether the drug inventory of pharmacy robots in each network is insufficient, and if so, sending a replenishment inventory request to the supply chain, and the supply chain platform replenishes inventory to the corresponding network pharmacy robot according to the replenishment inventory request, and according to changes in the disease spectrum of seasonal changes, forecasting the dynamic needs of drugs so that make the supply chain platform pre-stock demand to increase drug categories and remove them in time or reduce demand for shrinking drug categories.

A drug supplementation process 400 according to the embodiment of the present invention can comprise a plurality of methods as follows.

410: A cloud server statistics a data of a pharmacy robot device, the data includes a drug category, specification, dosage form, packaging size, drug supervision code to be replenished, and a category and quantity of nearly expired drugs.

420: A supply chain platform prepares the drugs according to the statistical data of the cloud server.

430: A logistics platform delivers the drugs to be replenished to the pharmacy robot device network.

440: A chassis brake of the pharmacy robot device unlocks, the pharmacy robot device goes to an open space, a connection between an edge shell and a central shell unlock, a left drug store and a right drug store and medicine box reveal, and then a manual replenishment method or a robot replenishment method can be used for replenishment.

442: The manual replenishment method includes a replenishment personnel scanning the medicine through a mobile smart terminal App or a visual sensor, a first indicator light of the medicine box corresponding to the medicine is on, showing a lack of medicine status, and guiding the replenishment personnel to find the medicine box lacking medicine in time and accurately, and according to a number of red lights, accurately make up for a shortage medicines, and repeat an operation to make up all the medicine boxes that are out of stock.

444: The robot replenishment method includes the replenishment robot arm equipped with suction cups and computer vision equipment, through a near field wireless communication component establishes a link with the pharmacy robot device, and an intelligent identification and intelligent control enable the suction cup to accurately identify the medicines to be replenished and place them in the medicine box that lacks medicines, and push the medicines to an appropriate distance, which is accurately calculated by an algorithm, and then the replenishment robot arm repeats the replenishment to replenish all out-of-stock medicine boxes.

450: The edge shell is coupled and locked with the central shell, moving the pharmacy robot device into place, and the chassis brake is locked.

A drug inventory process according to the embodiment of the present invention can comprise a drug inventory data collection and a drug inventory data statistical analysis. The drug inventory data can include a drug replenishment data, a drug sales data, and a drug near expiration date. The drug inventory data statistical analysis can sense a dynamic change of drug categories and quantities, submit replenishment requests in time, and predict future demand trends for medicines.

A grid deployment of an embodiment of the present invention can comprise a site selection, a pharmacy kiosk configuration, a single pharmacy robot or a pharmacy robot complex, and a drug category determination. A data sharing between the pharmacy kiosk and suppliers can make it easier for patients to obtain more drug information and obtain drugs and services through multiple channels.

A method for delivering medicines in a pharmacy robot complex according to the embodiment of the present invention can comprise a lifting and lowering a conveying device method and a lifting and lowering a collecting bin method, which are configured to lift medicines to a suitable height and solve a problem of a patient bending over to take medicines.

A lifting and lowering a conveying device method according to the embodiment of the present invention can include a screw device lifting and lowering method, a rope device lifting and lowering method, and a hydraulic device lifting and lowering method configured to lower a height of a convey device to a conveyor belt of the pharmacy robot, and undertake a medicine transferred by the conveyor belt, or raise the height of the convey device to a collection bin, and assist delivery of the medicine to the collection bin for a patient to take the medicine.

A lifting and lowering a screw device method according to the embodiment of the present invention can comprise an axially moving a screw in combination with a transport device and a screw lifting device method, and an axially moving a nut in with a transport device and a screw lifting device method. The axially moving a screw in combination with a transport device and a screw lifting device method includes: one end of a lead screw is fixed to the transport device, the other end is free, and a nut, an intelligent motor and a controller are fixed to a support part of the pharmacy robot body through a connecting piece, when the intelligent motor starts, the nut drives the lead screw to reciprocate, thereby realizing a lifting and lowering of the conveying device. The axially moving a nut in with a transport device and a screw lifting device method includes: one end of the lead screw is fixed on a support part of the pharmacy robot body, the other end is free, the nut, a intelligent motor and a controller are fixed on a transport device through a connecting piece, when the intelligent motor starts, the nut drives the conveying device to reciprocate, so as to realize the lifting and lowering of the conveying device.

A method for lifting and lowering a rope device according to the embodiment of the present invention can comprise a raising and lowering a conveying device combined with a pulley cable device method, and a raising and lowering a conveying device in combination with an axle cable device method. The raising and lowering a conveying device combined with a pulley cable device method can include: a driven wheel is connected to a transport device, a driving wheel is connected to an intelligent motor, one end of a rope is fixed to a support part of the pharmacy robot body, the driving wheel is connected to the driving wheel through the driven wheel, the intelligent motor is rotated forward, and the driving wheel is wound the rope to drive the driven wheel to rise, so that the conveying device rises, the intelligent motor rotates in the opposite direction, the driving wheel releases the rope, and drives the driven wheel to descend, so that the conveying device is lowered. The raising and lowering a conveying device in combination with an axle cable device method can include: two sets of wheel shaft rope devices are respectively fixed on a support parts of the pharmacy robot body, corresponding to both ends of a transport device, one end of a rope is connected to a driving wheel, and the other end is connected to the transport device, an intelligent motor of the rope device runs synchronously, and the driving wheel winds or releases the rope to drive the conveying device to rise or fall.

A method for lifting and lowering a hydraulic device method according to the embodiment of the present invention can comprise a conveying device combined with a hydraulic scissor lifting platform method, a conveying device combined with a single mast hydraulic lifting platform method, and a conveying device combined with a double mast hydraulic lifting platform method. A base of the hydraulic lifting platform can be arranged at a bottom of the pharmacy robot body, an upper part receives the conveying device, an intelligent vane pump is activated, and the lifting platform drives the lifting and lowering of the conveying device.

According to the embodiment of the present invention a collection bin lifting and lowering method can comprise a screw device lifting method, a rope device lifting method, and a hydraulic device lifting method, configured to lower a height of the collection bin to a conveyor belt or conveying device of the pharmacy robot, then the collection bin collects medicines, and then the collection bin raises to a height that is convenient for users to take the medicines.

A method for delivering medicines in a pharmacy robot complex according to the embodiment of the present invention can comprise a crawler method, a chute method, and an unmanned vehicle method, which is configured to solve a problem of medicine intercommunication and collection.

The crawler method for drug intercommunication and collection in a pharmacy robot complex according to the embodiment of the present invention can comprise a single-user service method and a multi-user parallel service method.

A single-user service of the crawler method according to the embodiment of the present invention includes:

(1) a medicine in a medicine box of the pharmacy robot complex reaches a crawler device through a conveyor belt, the crawler device starts, and runs reciprocate according to an algorithm, then the medicine arrives at a collection bin corresponding a user, and passes to the collection bin through a push device, and then the collection bin raises to a height of the user medicine-taking window; or

(2) a medicine in a medicine box of the pharmacy robot complex reaches a crawler device through a conveyor belt, and all the medicine can be gathered at one end of the crawler device, the crawler device runs to the a collection bin corresponding to a user, a push device pushes all the medicine to the collection bin, and then the collection bin raises to a height of the user's medicine taking window; or

(3) a medicine in a medicine box of the pharmacy robot complex reaches a crawler device through a conveyor belt, the crawler device carries the medicine, the crawler device rises to a height of the collection bin corresponding to a user, and then a push device pushes the medicine to a collection bin corresponding to the user.

A multi-user parallel service process 500 of a crawler method according to the embodiment of the present invention can comprise a plurality of steps as follows:

510: An algorithm calculates a time required for parallel users to take medicines, including a position of a single pharmacy robot where the medicines are located, a optimal route selection, and a quantity of medicines.

520: According to the time required sort the users, and prioritize the users with a least time required to serve the users, thereby reducing an overall waiting time of the users.

530: Complete a drug delivery of the users in sequence.

A chute method of an embodiment of the present invention can comprise a single-user service and a multi-user parallel service.

The single-user service process 600 of the chute method according to the embodiment of the present invention can comprise a plurality of steps as follows:

610: According to an algorithm, a robot arm accurately places a switch into a central chute, so that a proximal chute and a distal chute can be connected, and the distal chute can correspond to a medicine-taking service window selected by a user.

620: A medicine in a medicine box of the pharmacy robot complex reaches the proximal chute, the central chute and the far-end chute through a conveyor belt, and then enters a collection bin.

630: The collection bin raises to the medicine-taking service window for users to take the medicine.

The multi-user parallel service process 700 of the chute method according to the embodiment of the present invention can comprise a plurality of steps as follows:

710: Calculate a time required for users to take medicines according to an algorithm.

720: Rank the users according to the required time.

730: Priority service the user who requires a least time so as to reduce a overall waiting time.

740: Complete the user drug delivery in sequence.

An unmanned vehicle method according to the embodiment of the present invention can comprise a single-track unmanned vehicle method, a dual-track unmanned vehicle method, and a trackless unmanned vehicle method.

The single-track unmanned vehicle method of an embodiment of the present invention provides a single-user service can comprise a plurality of steps as follows:

(1) a medicine in a medicine box of the pharmacy robot complex arrives at a unmanned vehicle through a conveyor belt, the unmanned vehicle starts, and runs reciprocating according to an algorithm, and the medicine can be gathered to the unmanned vehicle, then the unmanned vehicle runs to a corresponding user's collection bin, and push the medicine to the corresponding user's collection bin through a push device, and then the collection bin raises to a height of the user's medicine-taking window; or

(2) a medicine in the medicine box of the pharmacy robot complex reaches a unmanned vehicle through a conveyor belt, the unmanned vehicle carries the medicine, then the unmanned vehicle and a track rises to a height of a collection bin corresponding user, and then the medicine can be pushed to the collection bin corresponding user through a push device.

The multi-user parallel service process 800 of the single-track unmanned vehicle method according to the embodiment of the present invention can comprise a plurality of steps as follows:

810: Calculate a time required for parallel users to take medicines according to an algorithm.

820: Rank the users according to the required time.

830: Priority service a user requiring a least time so as to reduce an overall waiting time of the user.

840: Complete a drug delivery of the user in sequence.

The single-user service of the dual-track unmanned vehicle method according to the embodiment of the present invention can comprise a plurality of steps as follows:

(1) a medicine in the medicine box of the pharmacy robot complex arrives at a unmanned vehicle through a conveyor belt, the unmanned vehicle starts, and according to an algorithm, two unmanned vehicles run back and forth, all the medicine can be gathered in the unmanned vehicle, and the unmanned vehicle then runs to a collection bin corresponding a user, a push device pushes all the medicines to the collection bin corresponding the user, and then the collection bin raises to a height of the user medicine-taking window; or

(2) a medicine in the medicine box of the pharmacy robot complex reaches a unmanned vehicle through a conveyor belt, the unmanned vehicle carries all the medicine, the unmanned vehicle and the track rise to a height of a collection bin corresponding a user, a push device pushes all the medicines to the collection bin corresponding the user.

The multi-user parallel service of the dual-track unmanned vehicle method according to the embodiment of the present invention can comprise a plurality of steps as follows:

(1) when serving two users, one unmanned vehicle serves one user respectively; or

(2) when serving three users, calculating a required time for the users to take medicines according to an algorithm, then the users are sorted according to the required time, and then priority service the user who takes a least time, thereby reducing an overall waiting time of the users and implementing medicine delivery.

A single-user service of a trackless unmanned vehicle method according to the embodiment of the present invention can comprise:

(1) each single pharmacy robot is equipped with an unmanned vehicle, a medicine arrives at a unmanned vehicle through a conveyor belt, the unmanned vehicle starts, and runs to a collection bin corresponding a user, a push device pushes the medicine to the collection bin corresponding the user, after the collection bin receives all the medicine, the collection bin raises to the user's medicine-taking window; or

(2) each single pharmacy robot is equipped with an unmanned vehicle, the unmanned vehicle carries all the medicine, the unmanned vehicle and a road platform rise to a height of a collection bin corresponding a user, a push device pushes the medicine to the collection bin corresponding the user.

The multi-user parallel service process 900 of the trackless unmanned vehicle method according to the embodiment of the present invention can comprise a plurality of steps as follows:

910: Configure one unmanned vehicle for each single pharmacy robot.

920: An algorithm calculates a required time for a user to take medicines.

930: According to the required time sort the user, prioritize the user with a least required time to serve so as to reduce an overall waiting time of the user.

940: Complete the medicine delivery in sequence.

A multiple unmanned vehicles serve a user process 1000 of an embodiment of the present invention can comprise a plurality of steps as follows:

1010: An unmanned vehicle configured by each single pharmacy robot is on standby at any time.

1020: Receive a medicine delivered by a conveyor belt of the single pharmacy robot.

730: When all the medicines the user needed arrive at the unmanned vehicle, the unmanned vehicle carries the medicine, starts and runs to a collection bin corresponding to the user.

1040: The unmanned vehicle without medicine continues to stand by.

1050: The medicine is pushed to the collection bin corresponding to the user.

1060: The collection bin raises to a height of the user medicine-taking window for the user to take medicine.

The multiple unmanned vehicles serving multiple users respectively process 1100 in the embodiment of the present invention can comprise a plurality of steps as follows:

1110: Configure one unmanned vehicle for each single pharmacy robot.

1120: Match each user with an exclusive unmanned vehicle.

1130: The exclusive unmanned vehicle is on call at any time to receive a medicine delivered by a conveyor belt of the single pharmacy robot.

1140: When all the medicine of the user arrive at the exclusive unmanned vehicle, the exclusive unmanned vehicle can run to a collection bin corresponding to the user.

1150: A push device Pushes the medicine to the collection bin corresponding to the user.

1160: The collection bin raises to the user medicine-taking window for the user to take medicine.

The multiple unmanned vehicles to cooperatively serve multiple users process 1200 in the embodiment of the present invention can comprise a plurality of steps as follows:

1210: Configure one unmanned vehicle for each single pharmacy robot.

1220: Match each user with two or more exclusive unmanned vehicles.

1230: The exclusive unmanned vehicle is on call at any time, and the exclusive unmanned vehicle is planned according to an algorithm and a principle of a least overall time consumption, and receive a medicine delivered by a conveyor belt of the single pharmacy.

1240: When all the medicines of the user need arrive at the exclusive unmanned vehicle, the exclusive unmanned vehicles run to a collection bin corresponding to the user.

1250: All the medicines on the exclusive unmanned vehicles are delivered to the collection bin corresponding to the user through a push device.

1260: The collection bin raises to the user medicine-taking window for the user to take medicine.

A system of an embodiment of the present invention can comprise an operating system, a software, an algorithms, a drug screening system, an AI verification system, an intelligent diagnosis and treatment system, a drug delivery system, a drug sales system, a drug management system, a third-party support system, a database, a data encryption system, a medical information sharing and supervision system, a drug circulation traceability system, an electronic prescription system, a cloud service, a 5G network, and a settlement system. The operating system includes a pharmacy robot device operating system and a Cartesian coordinate robot operating system. The software includes a pharmacy robot device software, a Cartesian coordinate robot software, and a mobile intelligent terminal application program. The algorithms include a Cartesian coordinate robot operation algorithm, a control algorithm, a drug big data analysis and prediction algorithm, an intelligent diagnosis and treatment, and a Deep Machine Learning Algorithms. The drug screening system can base on a disease spectrum and dynamic consumption data of a service population in different seasons of a service area, screen categories and quantities of drugs, appropriately increase the categories and quantities of drugs, and promptly reduce a number of drugs with shrinking demand. The AI verification system can be configured for drug category and dosage form verification, prescription verification, drug replenishment, and drug launch verification. The intelligent diagnosis and treatment system can traverse a latest clinical practice diagnosis and treatment operation specifications and medical knowledge map of human medicine, and extract feature values based on a structured, semi-structured and unstructured data of a patient main appeals, symptoms, signs, past medical history and examination results, with a help of artificial intelligence algorithms, to obtain clinical diagnosis and issue a treatment plan. The drug administration system includes a drug monitoring, a database data retrieval, a human-machine interaction or computer-computer interaction. The data encryption system can use blockchain technology to write patient data and drug data into blocks and publish them to public or private cloud servers. The pharmacy management system includes a drug supply chain management, a pharmacy management and pharmacy management, after a prescription is verified, the drug sales system can traverse the drugs in a drug store, if there is any stock, the Cartesian robot can execute a delivery, if there is no stock, the pharmacy robot can provide the patient with a drug arrival notification service, or notify a nearby outlets that have stock, or entrust a third-party logistics door-to-door drug delivery service. The third-party support system includes drug manufacturer, a drug distributor, a logistics, a drug regulatory agency, a communication service provider, and a third-party equipment maintenance. The drug circulation traceability system can adopt blockchain technology to monitor all links of drug circulation in real time until the patient takes it.

A system for delivering medicines in a pharmacy robot complex according to the embodiment of the present invention can comprise a pharmacy robot, a medical robot connected to the Internet of Things, a remote intelligent medical system, a mobile intelligent terminal, a processor, and a computer stored on a memory and running on the processor instructions, the computer instructions can be configured to execute by the processor, complete the delivery of the medicine by the pharmacy robot complex.

The computer-readable storage medium of one embodiment of the present invention stores instructions, programs, code sets, or instruction sets that can be loaded and executed by a processor to implement a single pharmacy robots and a pharmacy robot complex deliver medicines.

This application is intended to cover any variations, uses or adaptations of the invention which follow the general principles of the invention and which include common knowledge or conventional techniques in the art not disclosed by the invention. The description and examples are to be regarded as exemplary only, and the scope of the invention is limited only by the appended claims.

### INDUSTRIAL APPLICABILITY

(1) implementability: after reading an overall technical content disclosed in this application document, a person of ordinary skill in the technical field can objectively reproduce the subject matter of the invention-creation;

(2) reproducibility: according to the disclosed technical content, those of ordinary skill in the art can repeatedly implement the technical solutions adopted in this application to achieve its purpose. This repeated implementation has no quantitative limit and does not rely on any random factors, and each implementation yields the same result; and

(3) benefit: the pharmacy robot of the technical solution of the present application and the pharmacy robot complex constructed by the pharmacy robot can generate positive economic and social benefits.

What is claimed is:

1. A pharmacy robot device, having basic functions of pharmacy, comprising:
a single pharmacy robot configured to provide pharmacy as physician services, pharmacy as pharmacist services, and pharmacy as pharmacy affair management services, and
a pharmacy robot complex configured to provide pharmacy as physician services, pharmacy as pharmacist services, and pharmacy as pharmacy affair management services, the pharmacy robot complex comprises a plurality of the single pharmacy robots.

2. The pharmacy robot device according to claim 1, wherein the single pharmacy robot comprises:
a robot body comprises a shell, a drug store, a push rod, a conveyor belt, an induction door and a central data storage processor configured to implement a plurality of functions, and
a chassis configured to carry the robot body.

3. The pharmacy robot device according to claim 2, wherein the shell comprises:
a central shell configured to accommodate a drug store, and
a plurality of edge shells located on both sides of the central shell configured to accommodate the push rod.

4. The pharmacy robot device according to claim 3, wherein the drug store comprises a plurality of medicine cabinets, the medicine cabinet comprises a plurality of medicine boxes configured to store a medicine.

5. The pharmacy robot device according to claim 3, wherein the push rod comprises a plurality of Cartesian coordinate robots, the Cartesian coordinate robot includes an end effector configured to move a medicine in the drug store.

6. The pharmacy robot device according to claim 5, wherein the end effector comprises a servo motor, a reducer, a controller, a gear, a rack, and a guide rail, the guide rail is a semicircular grooved structure surrounding the gear and the rack.

7. The pharmacy robot device according to claim 1, wherein the pharmacy robot complex comprises:
a multi-unit pharmacy robot configured to provide pharmacy as physician services, pharmacy as pharmacist services, and pharmacy as pharmacy affair management services, and
a delivery device configured to connect the multi-unit pharmacy robot into an integral structure.

8. The pharmacy robot device according to claim 7, wherein the multi-unit pharmacy robot comprises a series pharmacy robots configured to provide medicines, the series pharmacy robots include a plurality of single pharmacy robots arranged in front and back, the single pharmacy robots share an induction door, and connect to each other with a conveyor belt.

9. The pharmacy robot device according to claim 8, wherein the multi-unit pharmacy robot comprises a parallel pharmacy robots configured to provide medicines to a plurality of patients in parallel, the parallel pharmacy robots include a plurality of single pharmacy robots arranged left and right, the single pharmacy robots connect to each other with a delivery device.

10. The pharmacy robot device according to claim 9, wherein the multi-unit pharmacy robot comprises a series-parallel pharmacy robots configured to provide a plurality of patients with a large variety of medicines in parallel, the series-parallel pharmacy robots include a plurality of single pharmacy robots arranged left and right, the single pharmacy robots connect to each other with the delivery device.

11. The pharmacy robot device according to claim 8, wherein the series-parallel pharmacy robots comprise a plurality of single pharmacy robots arranged in front and rear in series and left and right in parallel, the single pharmacy robots connect to each other with a delivery device.

12. The pharmacy robot device according to claim 11, wherein the delivery device comprises:
a conveying device includes a conveyor belt configured to convey a medicine, a collection bin connects to the conveying device configured to temporarily store the medicine, and
a lifting device configured to raise or lower the conveying device or the collection bin.

13. The pharmacy robot device according to claim 12, wherein the conveying device comprises a crawler device, and/or a chute device and/or an unmanned vehicle device, configured to connect a plurality of single pharmacy robots in parallel or in series or in series-parallel.

14. A pharmacy robot method, for realizing functions of pharmacy services, comprising:
selling a medicine, the medicine includes a drug, a medical equipment and a medical consumable,
supplementing the medicine,
inventorying the medicine,
conjoining a plurality of single pharmacy robots, and
grid deploying a pharmacy robot or pharmacy robot complex.

15. The pharmacy robot method according to claim 14, wherein the selling a medicine comprises:
pharmacy robot as physician services,
pharmacy robot as pharmacist services, and
pharmacy robot as pharmacy management services.

16. The pharmacy robot method according to claim 14, wherein the supplementing the medicine comprises:
processing data,
a supply chain platform preparing the medicine according to the processing data,
a logistics platform delivering the medicine to the pharmacy robot,
unlocking a brake of the pharmacy robot, and revealing a drug store, and
replenishing the medicine.

17. The pharmacy robot method according to claim 14, wherein the inventorying the medicine comprises:
collecting a medicine inventory data, and
analyzing the medicine inventory data.

18. The pharmacy robot method according to claim 14, wherein the conjoining a plurality of single pharmacy robots comprises:
arranging and combining a plurality of single pharmacy robots in front and back to form a series pharmacy robot complex, or
arranging and combining a plurality of single pharmacy robots left and right to form a parallel pharmacy robot complex, or
arranging and combining a plurality of single pharmacy robots in a front-rear series arrangement and a left-right parallel arrangement to form a series-parallel pharmacy robot complex.

19. The pharmacy robot method according to claim 14, wherein the grid deploying a pharmacy robot or pharmacy robot complex comprises:
selecting a site,
configuring a pharmacy kiosk, and
forming a single pharmacy robot or a pharmacy robot complex.

20. A pharmacy robot system, comprising:
a medicine screening system, an artificial intelligent verification system, an intelligent diagnosis and treatment system, a medicine delivery system, a medicine sales system, a pharmaceutical affairs management system, a third-party support system, a database management system, a data encryption system, a medical information sharing system and supervision system, a medicine circulation traceability system, an electronic prescription system, a cloud service, a 5G network, and a settlement system configured to support a pharmacy robot device to implement pharmacy functions.
